(19) United States
(12) Patent Application Publication
(10) Pub. No.: US 2016/0235347 A1
(43) Pub. Date: Aug. 18, 2016

(54) ARTIFICIAL SENSORS AND METHODS OF MANUFACTURE THEREOF

(71) Applicants: Maarij Baig, Waterloo (CA); Huayi Gao, Waterloo (CA); Kam Chiu Tam, Waterloo (CA); Vivek Maheshwari, Waterloo (CA); Xinyun Wu, Kitchener (CA)

(72) Inventors: Maarij Baig, Waterloo (CA); Huayi Gao, Waterloo (CA); Kam Chiu Tam, Waterloo (CA); Vivek Maheshwari, Waterloo (CA); Xinyun Wu, Kitchener (CA)

(21) Appl. No.: 15/043,537

(22) Filed: Feb. 13, 2016

Related U.S. Application Data
(60) Provisional application No. 62/115,886, filed on Feb. 13, 2015.

(51) Int. Cl. A61B 5/1486; C25D 3/48; C25D 5/54; A61B 5/145

(52) U.S. Cl. CPC A61B 5/1486; A61B 5/14532; C25D 3/48; C25D 5/54; A61B 2562/125

(57) ABSTRACT

Various embodiments are described herein for artificial sensors that have prolonged life-span and stability in harsh environments compared to sensors which use natural enzymes which easily denature under varying conditions. These sensors may be composed of novel nanostructures, and may be artificial sensors or artificial non-enzymatic sensors. In some embodiments, an artificial sensor may include a modulating and/or a cleansing electrode 4-electrode system.

### CROSS-REFERENCE TO RELATED APPLICATION

This application claims the benefit of U.S. Provisional Patent Application No. 62/115,886, filed Feb. 13, 2015; the entire contents of Patent Application No. 62/115,886 are hereby incorporated by reference.

### FIELD

Various embodiments are described herein generally relating to artificial analyte sensors (i.e. artificial sensors) and methods for manufacture thereof.

### BACKGROUND

The field of enzymatic sensors is rapidly growing to allow easier methods of preparation, simple immobilization routes, elimination of kinetic limitations and greater stability in different physiological conditions. The ability of enzymes to select for a specific compound has enabled use in biomedical, environmental and agricultural fields. However, current trends are for the use of sensors in physiological conditions. For example, considering that modern diabetes management is moving towards standardizing continuous glucose monitoring (CGM), a glucose sensor that is functional in physiological conditions for long term use would be very advantageous. Alternatively, for other applications such as, but not limited to, treating inflammation (e.g. in the eye or in other body parts), eye health (e.g. interleukin 1, Interleukin 6), neurodegenerative diseases (e.g. Alzheimer's), metabolic diseases, and degenerative central nervous system disorders (e.g. Parkinson's disease), sensors are needed for use in physiological conditions. However, enzymatic-based sensors degrade under physiological conditions.

### SUMMARY OF VARIOUS EMBODIMENTS

Various embodiments for artificial sensors and associated methods of manufacture are provided according to the teachings herein. Various modifications may be made so that the artificial sensor retains at least one of its sensitivity, specificity, and stability in nominal physiological conditions as well as in harsh conditions. In some cases, in accordance with the teachings herein, a 4-electrode system may be used for the artificial sensor to facilitate better sensing performance for various target analytes) of interest.

In a broad aspect, at least one embodiment described herein provides an artificial sensor for sensing target analytes, wherein the artificial sensor may comprise a working electrode that is configured to provide a detection signal indicating detection of the target analytes during a sensing phase; a reference electrode that is configured to provide a reference level for measurements made at the working electrode; a counter electrode that is configured to provide a current source or a current sink for the working electrode during use; and at least one additional electrode that is configured to improve signal to noise ratio of the detection signal when provided with a control voltage during use.

In at least some embodiments, the at least one additional electrode may comprise a modulating electrode that is configured to modify local conditions around the working electrode when provided with the control voltage during use.

In at least some embodiments, the modulating electrode may be configured to increase a number of rate-limiting reagents in the micro-environment when provided with the control voltage during use.

In at least some embodiments, the rate-limiting reagents may comprise at least one of O₂, H₂, H₂O₂, H₂O, and OH.

In at least some embodiments, the modulating electrode may be configured to generate a desired local pH in the micro-environment by consuming or producing hydroxide when provided with the control voltage during use.

In at least some embodiments, the control voltage may comprise sequences of electrical pulses at different voltage, current, or charge conditions to temporarily modify the local conditions of the micro-environment.

In at least some embodiments, the control voltage may be applied to the modulating electrode before the equilibrium is achieved at the working electrode and before the sensing phase for detection of target analytes.

In at least some embodiments, the control voltage may be removed anytime between the beginning and end of the sensing phase.

In at least some embodiments, the at least one additional electrode may comprise a cleansing electrode that is configured to breakdown or consume an interference species when the control voltage is applied during the sensing phase to reduce an effect of the interference species on the working electrode.

In at least some embodiments, the cleansing electrode may comprise artificial enzymes or targeting sites to breakdown the interference species.

In at least some embodiments, the voltage, current or charge of the cleansing electrode may be varied during use to cleanse different interference species sequentially or simultaneously.

In at least some embodiments, the cleansing electrode may be configured to receive a charge to attract interference species and to repel target analytes.

In at least some embodiments, a surface area of the cleansing electrode may be increased to draw away more interference species from the working electrode.

In at least some embodiments, the cleansing electrode may be configured to provide a catalyzing activity for the interference species and enable regeneration of active sites for the cleansing electrode.

In at least some embodiments, the cleansing electrode may be configured to convert an interference species to a non-interference species by selectively oxidizing or reducing the interference species.

In at least some embodiments, the cleansing electrode may comprise a conducting polymer that acts as a reducing agent to decrease the effect of the interference species on the working electrode.

In at least some embodiments, the cleansing electrode may be placed on a same plane as the working electrode.

In at least some embodiments, the at least one additional electrode may comprise a modulating electrode and a cleansing electrode wherein the modulating electrode may be configured to modify local conditions around the working electrode when provided with the control voltage during use and the cleansing electrode may be configured to breakdown or consume an interference species when the control voltage is applied during the sensing phase to reduce an effect of the interference species on the working electrode.

In at least some embodiments, a regeneration voltage waveform may be applied to the working electrode after the end of the sensing phase to remove adsorbed species from a surface of the working electrode.

In at least some embodiments, the regeneration voltage waveform may not be applied after the end of every sensing phase.

In another broad aspect, at least one embodiment described herein provides an artificial sensor for sensing target analytes, wherein the artificial sensor may comprise a working electrode that is configured to provide a detection signal indicating detection of the target analytes during a sensing phase and to receive a control voltage during a modulation phase to modify local conditions around the working electrode to improve signal to noise ratio of the detection signal; a reference electrode that is configured to provide a reference level for measurements made at the working electrode; and a counter electrode that is configured to provide a current source or a current sink for the working electrode during use.

In at least some embodiments, the working electrode may be configured to operate as the modulating electrode as defined in accordance with the teachings herein.

In another broad aspect, at least one embodiment described herein provides a method of sensing target analytes using an artificial sensor, wherein the method may comprise generating a detection signal at a working electrode indicating detection of the target analytes thereabout during a sensing phase; providing a reference level at a reference electrode for providing a baseline for measurements made at the working electrode; providing a current source or a current sink at a counter electrode to provide or remove current from the working electrode during use; and applying a control voltage to at least one additional electrode to improve signal to noise ratio of the detection signal.

In at least some embodiments, the method may comprise using a modulating electrode as the at least one additional electrode to modify local conditions around the working electrode when provided with the control voltage.

In at least some embodiments, the method may comprise using the modulating electrode to increase a number of rate-limiting reagents in the micro-environment when provided with the control voltage during use.

In at least some embodiments, the method may comprise applying the control voltage to the modulating electrode to generate a desired local pH in the micro-environment by consuming or producing hydroxide.

In at least some embodiments, the method may comprise applying sequences of electrical waveforms at different voltage, current, or charge conditions in the control voltage to temporarily modify the local conditions of the micro-environment.

In at least some embodiments, the method may comprise applying the control voltage to the modulating electrode before equilibrium is achieved at the working electrode and before the sensing phase for the detection of target analytes.

In at least some embodiments, the method may comprise removing the control voltage anytime between the beginning and end of the sensing phase.

In at least some embodiments, the method may comprise using a cleansing electrode for the at least one additional electrode to breakdown or consume an interference species when the control voltage is applied during the sensing phase to reduce an effect of the interference species on the working electrode.

In at least some embodiments, the method may comprise varying the voltage, current or charge of the cleansing electrode during use to cleanse different interference species sequentially or simultaneously.

In at least some embodiments, the method may comprise providing a charge at the cleansing electrode to attract interference species and to repel target analytes from the cleansing electrode.

In at least some embodiments, the method may comprise increasing a surface area of the cleansing electrode to draw away more interference species from the working electrode.

In at least some embodiments, the method may comprise providing a catalyzing activity at the cleansing electrode for the interference species and enabling regeneration of active sites for the cleansing electrode.

In at least some embodiments, the method may comprise using the cleansing electrode to convert an interference species to a non-interference species by selectively oxidizing or reducing the interference species.

In at least some embodiments, the method may comprise using a modulating electrode and a cleansing electrode for the at least one additional electrode wherein the modulating electrode may be used to modify local conditions around the working electrode when provided with the control voltage during use and the cleansing electrode may be used to breakdown or consume an interference species when the control voltage is applied during the sensing phase to reduce an effect of the interference species on the working electrode.

In at least some embodiments, the method may comprise applying a regeneration voltage waveform to the working electrode after the end of the sensing phase to remove adsorbed species from a surface of the working electrode.

In at least some embodiments, the regeneration voltage waveform may not be applied after the end of every sensing phase.

In another broad aspect, at least one embodiment described herein provides a working electrode for an artificial sensor that detects target analytes, wherein the working electrode comprises a base electrode that is conductive; an intermediary layer disposed adjacent to the base electrode; comprising growth sites; and an artificial functional layer that is coupled to the intermediary layer and configured to provide an artificial sensing function, the artificial functional layer comprising at least one artificial catalyst.

In at least some embodiments, the intermediary layer may comprise a fractal metal nanostructure that provides a template with increased surface area for the at least one artificial catalyst in the functional layer.

In at least some embodiments, the fractal structure may comprise one or more metals.

In at least some embodiments, the artificial functional layer may comprise a multi-metallic nanostructure where the nanostructure enhances working electrode performance.

In at least some embodiments, the artificial functional layer may be configured for detecting a desired type of target analyte at a desired detection sensitivity and detection specificity by including multiple metals or metal alloys into the artificial functional layer.

In at least some embodiments, the functional layer may comprise platinum nanostructures that detect glucose.

In at least some embodiments, the intermediary layer may comprise a metal chelating polymer and a carbon nanomaterial, wherein the metal chelating polymer covers the carbon nanomaterial and the carbon nanomaterial is doped or un-doped.

In at least some embodiments, the metal chelating polymer may comprise a conductive polymer.

In at least some embodiments, the metal chelating polymer may comprise an insulating polymer that is thin enough to allow electron transfer thereacross.

In at least some embodiments, the insulating polymer may comprise polydopamine.

In at least some embodiments, the intermediary layer may comprise nanoparticles that are produced on the metal chelating polymer, are dense and are similar in size range to provide a more homogeneous distribution of catalyst sites in the functional layer and improve detection.

In at least some embodiments, the nanoparticles may comprise gold nanoparticles having a size in the range of about 3-8 nm to detect glucose.

In at least some embodiments, the metal chelating polymer may comprise at least one of polypyrrole (PPY), Polydopamine, Poly-thiophenes (PEDOT) and its derivatives, Polyaniline (PANI) and its derivatives, Poly(para-phenylene Vinylene) (PPV), Poly(Carbazole), Polyacetylene, and Polyfuran.

In at least some embodiments, the intermediary layer may comprise PPY-CNC or PDA-CNC disposed adjacent to the base electrode.

In another broad aspect, at least one embodiment described herein provides a method for creating an artificial sensor for detecting target analytes, wherein the method comprises: creating a base electrode; creating an intermediary layer on the base electrode; and creating an artificial functional layer on the intermediary layer.

In at least some embodiments, the method may comprise growing a fractal metal nanostructure in the intermediary layer to provide a template with increased surface area for at least one artificial catalyst in the functional layer.

In at least some embodiments, the method may comprise growing a multi-metallic structure in the artificial functional layer to improve working electrode performance by performing a second growth step through electrodeposition of a secondary growth solution.

In at least some embodiments, the additional layers may be grown from at least one of Au, Ag, Ti, Al, Pt, Cu, Ni, alloy, a conductive polymer, and metal oxide.

In at least some embodiments, the method may comprise growing platinum nanostructures in the artificial functional layer to detect glucose.

In at least some embodiments, the act of creating the intermediary layer may comprise selecting a doped or undoped carbon nanomaterial; and coating the carbon nanomaterial with a chelating metal polymer.

In at least some embodiments, coating the carbon nanomaterial may comprise providing an insulating polymer that is thin enough to allow electron transfer thereacross.

In at least some embodiments, the act of creating the artificial functional layer may comprise forming metal particles having catalyst functionality in the artificial functional layer.

In at least some embodiments, the act of forming metal particles may comprise adding a metal precursor with a reducing agent or an oxidizing agent.

In at least some embodiments, the method may further comprise adding a protective coating to the artificial sensor to reduce a number of common interferences at the artificial sensor.

In at least some embodiments, creating the intermediate layer may comprise selecting a conducting polymer for integration in a Cellulose Nano-Crystal (CNC) hybrid structure.

In at least some embodiments, creating the artificial functional layer may comprise adding material to a solution to provide enzyme mimicking behaviour to the hybrid structure.

In at least some embodiments, the additional material may comprise one of Au, Ag, Ti, Al, Pt, Cu, Ni, metal alloys, conductive polymer, metal oxide and carbon based material.

In at least some embodiments, the carbon based material may comprise one of reduced graphene oxide, graphene, fullerene, and a Multi-wall Carbon Nanotube (MWCNT).

In at least some embodiments, the fractal metal nanostructure may comprise a gold nanostructure, a surface area of active sites on the functional layer is limited, and a ratio of a volume of the gold nanostructure to the limited surface area is selected to affect the kinetics of an analyte reaction with the target analytes.

In another broad aspect, at least one embodiment described herein provides a method of sensing target analytes using an artificial sensor, wherein the method may comprise providing a detection signal at a working electrode indicating detection of the target analytes during a sensing phase; providing a reference level at a reference electrode for measurements made at the working electrode; and providing a current source or a current sink at a counter electrode for current flow with the working electrode during use; and providing a control voltage at the working electrode during a modulation phase to modify local conditions around the working electrode to improve signal to noise ratio of the detection signal.

In at least some embodiments, the working electrode may be configured to operate as the modulating electrode defined in accordance with the teachings herein.

In another broad aspect, at least one embodiment described herein provides a working electrode for an artificial sensor that detects target analytes, wherein the working electrode may comprise a base electrode that is conductive; an intermediary layer disposed adjacent to the base electrode; comprising a conductive metal chelating polymer-cellulose nano crystal structure (CNC); and an artificial functional layer that is coupled to the intermediary layer and configured to provide an artificial sensing function, the artificial functional layer comprising a nanoparticle structure that mimic enzyme activity for detecting the target analytes.

In at least some embodiments, the nanoparticle structures may comprise nanoparticles having a specified size and shape to provide a specified function.

In at least some embodiments, the nanoparticles may be specified to detect glucose.

In at least some embodiments, the nanoparticles may comprise gold nanoparticles having a size of about 30 nm to provide to provide a specified function.

In at least some embodiments, the metal chelating polymer may comprise at least one of polypyrrole (PPY), Poly-thiophenes (PEDOT) and its derivatives, Polyaniline (PANI) and its derivatives, Poly(para-phenylene Vinylene) (PPV), Poly(Carbazole), Polyacetylene, and Polyfuran.

In at least some embodiments, the intermediary layer and the artificial functional layer may be provided by CNC-PPY disposed adjacent to the base electrode.

In at least some embodiments, a filler compound may be added to nanoparticles in the artificial functional layer to allow for printing the artificial functional layer on a substrate.

In at least some embodiments, the working electrode may comprise a protective coating on the artificial functional layer to minimize interaction with common interferences to promote increased detection sensitivity.

In at least some embodiments, the protective coating may comprise one of a positively or negatively charged polymer, a microporous membrane, an anionic or cationic hydrogel, and a perflourinated membrane.

In at least some embodiments, subunits for the nanoparticle structure may be selected to bind with and detect the target analytes.

In another broad aspect, at least one embodiment described herein provides a method for creating an artificial sensor for detecting target analytes, wherein the method may comprise selecting a metal chelating polymer for integration in a Cellulose Nano-Crystal (CNC) hybrid structure; adding material to a solution to provide enzyme mimicking behaviour to the hybrid structure, the enzyme mimicking behaviour being chosen depending on the target analytes; applying the solution to a working electrode of the artificial sensor; and adding a protective coating to the artificial sensor to reduce a number of common interferences at the artificial sensor.

In at least some embodiments, the solution may be drop cast onto the working electrode.

In at least some embodiments, the target analytes may comprise glucose the method may further comprise adding peroxidase mimicking metal salt to the solution after adding the material providing the enzyme mimicking behaviour.

In at least some embodiments, the metal chelating polymer may comprise one of PPY, Poly-thiophenes (PEDOT) and its derivatives, Polyaniline (PANI) and its derivatives, Poly(para-phenylene Vinylene) (PPV), Poly(Carbazole), Polyacetylene, and Polyfuran.

In another broad aspect, at least one embodiment described herein provides a working electrode for an artificial sensor that detects target analytes, wherein the working electrode may comprise a base electrode that is conductive; and an artificial functional layer that is coupled to the base electrode and is configured to provide an artificial sensing function, the artificial functional layer comprising a fractal structure for performing a certain function.

In at least some embodiments, the fractal structure may be grown on a selected crystal plane of a base crystal structure that is in the artificial functional layer.

In at least some embodiments, the fractal structure may comprise one or more metals.

In another broad aspect, at least one embodiment described herein provides a working electrode for an artificial sensor that detects target analytes, wherein the working electrode may comprise a base electrode that is conductive; and an artificial functional layer that is coupled to the base electrode and is configured to provide an artificial sensing function, the artificial functional layer comprising a multi-metallic nanostructure where the nanostructure enhances working electrode performance.

In at least some embodiments, the nanostructure may comprise nanoparticles arranged in a certain way to mimic the function of a desired enzyme.

In at least some embodiments, the nanostructure may comprise nanoparticles arranged in different ways to mimic the function of different enzymes.

In at least some embodiments, sensing activity for the working electrode may be determined by favorable active sites in the artificial functional layer that provide more efficient catalytic activities.

In at least some embodiments, the artificial functional layer may comprise an artificial enzyme fabricated by using one of electroplating, chemical synthesis, photolithography and e-beam lithography.

In at least some embodiments, the artificial enzyme may comprise periodic nanostructures including one of fractal structures, nano-plates, nano-pillars, nano-wires and nano-particles.

In another broad aspect, at least one embodiment described herein provides a method for creating an artificial sensor for detecting target analytes, wherein the method may comprise preparing a gold salt growth solution with an added electrolyte to grow fold fractal nanostructures; defining a limited surface area of active sites on a working electrode of the artificial sensor by using a cylindrical well of a liquid holder; adding the gold salt growth solution to the cylindrical well; submerging a counter electrode and a reference electrode in the cylindrical well; coupling electrical leads to the working electrode, the counter electrode and the reference electrode; applying a potential to the gold growth solution for a certain amount of time depending on material used for the working electrode and a desired morphology for the nano-structure; and applying a protective coating to the artificial sensor to minimize common interferences in a surrounding environment from fouling the artificial sensor.

In at least some embodiments, a second growth step may be performed for creating a bi-metallic or a multi-metallic structure through electrodeposition of a secondary growth solution.

In at least some embodiments, the additional layers that are grown may be made from at least one of Au, Pd, Ag, Pt, Cu, CuO, NiOH, and Ni on top of the current material.

In at least some embodiments, the surface area of the active sites may be limited by restricting growth of the gold fractal nanostructure to the limited surface area on the working electrode.

In at least some embodiments, the act of coupling electrical leads may comprise coupling a first lead from a potentiostat to a protruding surface on the working electrode where the protruding surface is not exposed to the gold growth solution and coupling second and third leads from the potentiostat to the counter electrode and the reference electrode.

In at least some embodiments, a ratio of a volume of the gold nanostructure to the limited surface area may be selected to affect the kinetics of an analyte reaction with the target analytes.

In at least some embodiments, the counter electrode may comprise a platinum wire and the reference electrode may comprise a Ag/AgCl electrode.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described.

FIG. 1A is a flowchart of an example embodiment of an electrode fabrication method that may be used to fabricate a working electrode for an artificial sensor.

FIG. 1B is a flowchart for providing examples of implementations that may be used in the electrode fabrication method of FIG. 1A.

FIG. 2A is a schematic showing various alternatives for fabricating artificial sensors for use in various applications.

FIG. 2B is a schematic drawing showing an example of a fractal structure that may be used in an artificial functional layer of an artificial sensor.

FIG. 3A is a flowchart of an example embodiment of an artificial enzymatic sensor fabrication method to fabricate a hybrid carbon-metal based sensor;

FIG. 3B is a flowchart of an example embodiment of an artificial enzymatic sensor fabrication method for fabricating an artificial enzymatic sensor.

FIGS. 4A-4F are TEM images of (a) starting carbon material (b) nitrogen doped carbon precursors (c) nitrogen doped carbon (d) nitrogen doped carbon coated with metal chelating polymer (e) nitrogen doped carbon functionalized with Pt particles and (f) nitrogen doped carbon functionalized with Pd particles.

FIG. 4G shows thermal analysis results of Thermal Gravimetric Analysis (TGA) for Pt—N-PDCNC and Pd—N-PDCNC.

FIG. 5A is a flowchart of an example embodiment of an artificial enzymatic sensor fabrication method for fabricating an artificial enzymatic sensor that uses a fractal gold structure in the artificial functional layer.

FIG. 5B is a schematic drawing showing an example embodiment of a synthesis apparatus that may be used to make some of the artificial sensors described in accordance with the teachings herein.

FIG. 6A is a schematic drawing showing an example embodiment of an electrode system for an artificial sensor where the electrode system may include up to five electrodes.

FIG. 6B is a top view showing the layout of another example embodiment of an artificial sensor having a cleansing electrode.

FIG. 6C is a top view showing the layout of another example embodiment of an artificial sensor having a modulating electrode.

FIGS. 6D and 6E are top and cross-sectional views, respectively, showing the layout of another example embodiment of an artificial sensor having a cleansing electrode and a modulating electrode.

FIGS. 7A and 7B are schematic drawings showing the operation between a modulating electrode and a working electrode of an artificial sensor.

FIGS. 8A to 8C are schematic drawings showing the operation between a cleansing electrode and a working electrode for an artificial sensor.

FIG. 9 shows example plots for voltage, current and collected data for an example embodiment of a detection scheme used with a 3-electrode artificial sensor.

FIG. 10 shows example plots for voltage, current and collected data for an example embodiment of a detection scheme used with a 4-electrode artificial sensor including a modulating electrode.

FIG. 11 shows example plots for voltages and collected data for an example embodiment of a detection scheme used with a 4-electrode artificial sensor including a modulating electrode for regeneration and pH modulation.

FIG. 12 shows example plots for voltage, current and collected data for an example embodiment of a detection scheme used with a 3-electrode artificial sensor where the working electrode is used for sensing and modulation.

FIG. 13 shows an example of an Amperometric detection curve for the detection of glucose using a fractal gold structure in neutral pH conditions.

FIG. 14 shows an example of an Amperometric detection curve using a fractal gold structure in alkaline pH conditions (PBS +0.1 M NaOH).

Further aspects and features of the embodiments described herein will appear from the following description taken together with the accompanying drawings.

### DETAILED DESCRIPTION OF THE EMBODIMENTS

Various apparatuses or processes will be described below to provide an example of at least one embodiment of the claimed subject matter. No embodiment described below limits any claimed subject matter and any claimed subject matter may cover processes, sensors or devices that differ from those described below. The claimed subject matter is not limited to processes, sensors or devices having all of the features of any one process or device described below or to features common to multiple or all of the processes, sensors or devices described below. It is possible that a process or device described below is not an embodiment of any claimed subject matter. Any subject matter that is disclosed in a process, sensor or device described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical element or electrical signal (either wired or wireless) or a mechanical element depending on the particular context or via chemical bonds such as ionic bonds, hydrogen bonds, Vanderwaal interaction, covalent bonds and the like.

It should be noted that terms of degree such as “substantially”, “about” and “approximately” as used herein mean a reasonable amount of deviation of the modified term such that the end result is not significantly changed. These terms of degree may be construed as including a certain deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both X and Y, for example. As a further example, “X, Y, and/or Z” is intended to mean X or Y or Z or any combination thereof.

Artificial sensors can function long term in physiologically relevant conditions. However, conventional artificial sensors suffer from other issues depending on the application. For example, conventional artificial sensors suffer from one or more of: i) poor specificity towards biomarkers (such as glucose for example), ii) poor sensitivity, iii) poisoning by interferences, iv) requiring a high working potential and v) stringent pH requirements.

Described herein are various example embodiments for an artificial sensor that generally comprise multi-metallic nano-structures, which are desirable for their prolonged life-span and stability in harsh environments compared to sensors which use natural enzymes which easily denature under varying conditions (e.g. temperature, pH, etc.). Sensors composed of novel nanostructures, in accordance with the teachings herein, may therefore have enhanced sensitivity, specificity, and stability.

In some cases, the artificial sensors described in accordance with the teachings herein may be referred to as artificial enzymatic (i.e. enzyme mimicking) sensors since they display the properties of an enzyme that naturally catalyzes a reaction to detect a target molecule or a target compound (hereafter both referred to as a target analyte). However, in other cases the artificial sensors described in accordance with the teachings herein may be referred to as artificial non-enzymatic sensors that detect target analytes for which there are no naturally occurring enzymes that catalyze reactions to detect the target analyte.

In alternative embodiments, in accordance with the teachings herein, an artificial sensor may include a 4-electrode system which operates similarly to standard 3-electrode systems and comprises the W.E., C.E. and R.E., but also harnesses added benefits from using a 4^{th} electrode which may provide various functions such as, but not limited to, providing a modulating function and/or a cleansing function. In some cases, the artificial sensor may use a 5 electrode system which includes a modulating electrode and a cleansing electrode. Each additional electrode in the 4-electrode or 5-electrode systems aims to increase the sensed signal, while minimizing the noise signal, which is beneficial for sensor performance when analytes are present in extremely low concentrations. In some embodiments, a 3-electrode artificial sensor, comprising a working electrode (W.E.), a counter electrode (C.E.) and a reference electrode (R.E.), may be modified so that the W.E. is used for sensing and micro-environment modulation in accordance with the teachings herein.

In some embodiments, it may also be possible to use one electrode to provide both the modulating and cleansing functions. This may be done in cases where the species that is produced by modulating may also allow the W.E. to selectively bind the target molecule and also repel interference species and thus providing a cleansing function.

Artificial enzymes excel in their stability over natural enzymes such as glucose oxidase and peroxidase. On top of that, artificial enzymes are more robust as they can range in structure from 0D to 3D nanostructures such as, but not limited to, nano-particles, nano-rods, nano-wires, nano-sheets, nano-clusters, nano-branches, nano-thorns, and nano-belts, for example. Since structure suggests function, being able to use a variety of different nanostructures in the artificial sensors described in accordance with the teachings herein translates to being able to provide a variety of different functions, which is advantageous. In addition, the choice of distinct metal materials and combination of multi-metal composites provides extra dimensions of freedom for the creation of desirable artificial behaviors.

It should be noted that the various artificial sensors described in accordance with the teachings herein may be used to sense biological analytes in which case the artificial sensors may be referred to as artificial biosensors. Accordingly, artificial enzymatic sensors and artificial non-enzymatic sensors that sense biological analytes may be referred to as artificial enzymatic biosensors and artificial non-enzymatic biosensors.

In at least one embodiment in accordance with the teachings described herein, a carbon-metal hybrid based artificial sensor may be created that can provide a non-enzymatic or enzymatic mechanism for certain markers. For example, a metal chelating polymer-carbon nanomaterial based artificial sensor that can provide a non-enzymatic or enzymatic mechanism to certain markers (e.g. target analytes) may be used to sense various molecules and compounds including biomarkers, for example. The carbon nanomaterial candidates include, but are not limited to, cellulose nanocrystals (CNC) and its derivatives, graphene and its derivatives, carbon nanotubes and its derivatives, and fullerenes and its derivatives. Cellulose Nanocrystals (CNC) is an advantageous carbon material as it is biocompatible and a stable template material. A metal chelating polymer-CNC (MC-CNC) based artificial enzymatic biosensors may use 0D, 1D, 2D, or 3D nanostructures to provide its functions towards biomarkers such as, but not limited to, glucose, lactate, and uric acid, for example. Poly-Pyrrole (PPY) is a type of conductive polymer, and is only one example of many conductive polymer candidates that may be used with a carbon material such as CNC to create PPY-CNC. Polydopamine (PDA) is a type of insulating polymer, and is only one example of many insulating polymer candidates that may be used with a carbon material such as CNC to create PDA-CNC. These polymer candidates include, but are not limited to, Poly-thiophenes and its derivatives, Polydopamine (PDA) and its derivatives, Polyaniline (PANI) and its derivatives, Poly(para-phenylene Vinylene) (PPV), Poly(Carbazole), Polyacetylene, and Polyfuran.

In at least some embodiments, various functional groups such as, but not limited to, Au, Ag, Ti, Al, Pt, Cu, Ni, metal alloys, conductive polymer, metal oxide (ZnO, CuO, Cu2O), and carbon-based materials (e.g. reduced graphene oxide, Multi-wall Carbon Nanotube (MWCNT)) may also be grafted onto the surface of the MC-CNC platform. For example, if detection of multiple analytes is required, then corresponding 0D, 1D, 2D, or 3D nano-structures that have these sensing capabilities may be immobilized either covalently or non-covalently to the MC-CNC platform.

Other functional groups designed to minimize interference with non-target molecules may also be grafted on the same platform to improve sensitivity in at least some embodiments. For example, some functional groups can be used to increase reactivity with interferences, thereby eliminating them from the solution. Such functional groups include, but are not limited to, acyl, carboxyl, silyl, PEG (poly-ethylene glycol) grafted, PEO (poly(ethylene oxide)), PCL (Poly(caprolactone)), PLA (poly(lactic acid)), and PMMAZO (poly[6-(4-methoxy-azobenzene-4′-oxy) hexylmethacrylate]), PS (poly(styrene), PAA (Poly(acrylic acid)), and PDMAEMA (Poly(2-(dimethylamino)ethylmethacrylate)), for example.

A 0D nano-structure may include nanoparticles that have dimension ranging from 1 nm to 100 nm. An example of using 0D nano-structures for analyte detection is the addition of nanoparticles to PPY-CNC. A 1D nano-structure may include rods, belts, or tubes having one dimension with a size not in the range of 1 nm to 100 nm. A 2D nano-structure may include sheets of material in which either dimension (i.e. the length or width) do not fall within the range of 1 nm to 100 nm. A 3D nano-structure has a more complex shape in which none of the dimensions fall within the range of 1 nm to 100 nm. An example of a 3D nano-structure is a fractal gold pattern.

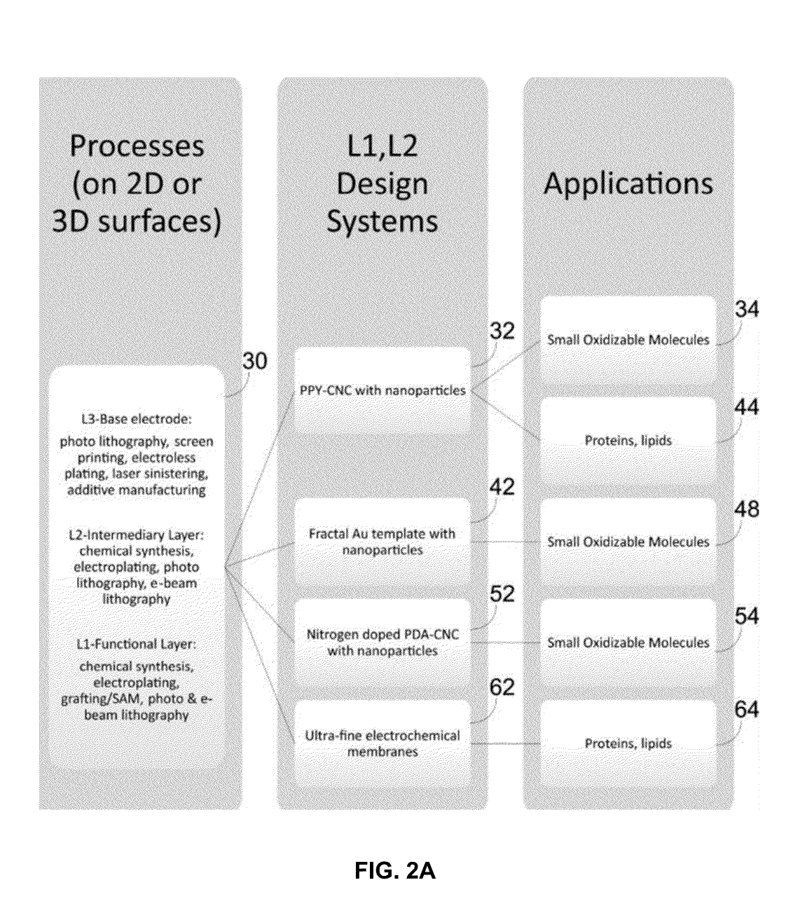

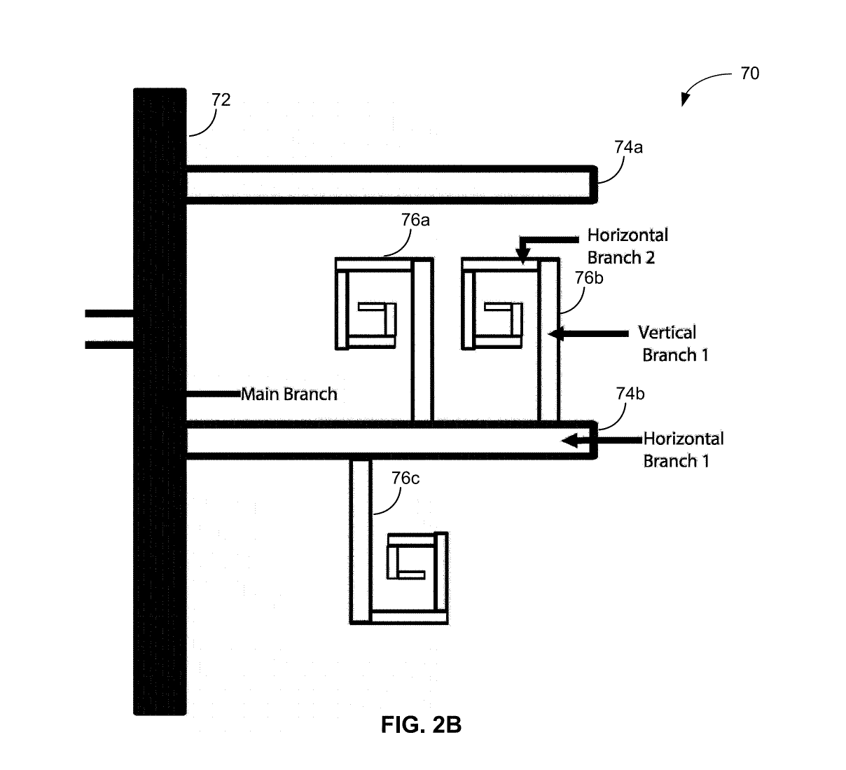

In at least one embodiment described in accordance with the teachings herein, an artificial sensor may be a multi-metallic fractal nano-composite biosensor. A fractal composite is a fractal-like 3D structure that has a repeating structural similarity on a wide range of sizes (an example of which is shown in FIG. 2B). Due to its high complexity, extremely high surface areas are present, especially when nano-scale fractal composites are created. Such a compact, yet convoluted structure provides countless defect sites for the catalytic reaction of target molecules such as glucose, for example. In some cases it may be possible to enhance these types of artificial sensor by targeting specific nanostructures in the fabrication process for highly uniform structures, with enhanced specificity.

In at least some embodiments described in accordance with the teachings herein, for artificial sensors that use a 4-electrode system or a 5-electrode system having at least one of a modulating electrode and a cleansing electrode, various voltage, current and/or charge schemes may be used to provide certain functions to achieve an improvement in performance over that of 3-electrode systems and/or to adapt the 4 or 5 electrode systems for use in certain applications.

For example, in some embodiments, a modulating electrode may be added to 3 or 4 electrode systems to modify local conditions in the micro-environment around the artificial sensor by creating an abundance of rate-limiting reagents such as oxygen or by changing the local pH by consumption or production of hydroxide to prime the W.E., for example. However, this temporary micro-environment quickly dissipates as system equilibrium is restored over time, or as all the produced species are consumed. Artificial sensor activity may be enhanced within these micro-environments.

In alternative embodiments, a cleansing electrode may be added to 3 or 4 electrode systems to eliminate interference species, by using oxidation or decomposition. For example, in some embodiments, the surface of the cleansing electrode may also include artificial enzymes or reactive species that target specific interference molecules.

In alternative embodiments, a modulating electrode and a cleansing electrode may be added to a 3 electrode sensor system to improve performance. Thus, the addition of the modulating and cleansing electrodes serve to further improve sensing performance as compared to when these additional electrodes are used separately.

### Creation of a Working Electrode

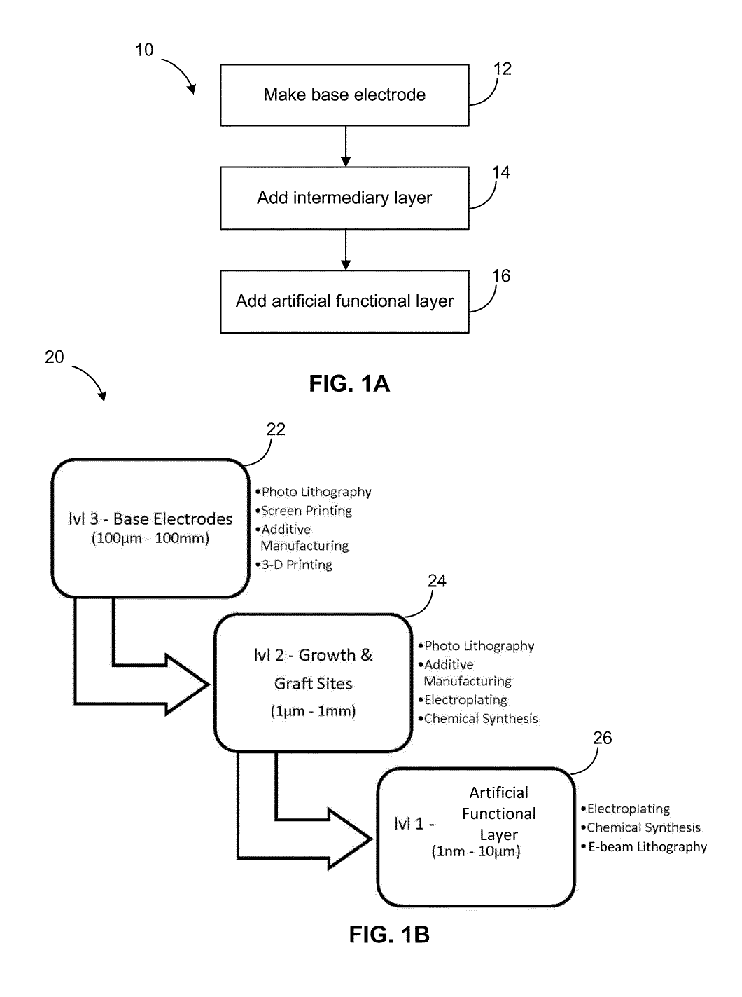

Referring now to FIG. 1A, shown therein is a flowchart of an example embodiment of an electrode fabrication method 10 that may be used to fabricate a working electrode for an artificial sensor in accordance with the teachings herein. The method 10 may be done in a clean room environment. The electrode fabrication method 10 may be used to fabricate other types of electrodes such as the C.E., the R.E., modulating electrodes and cleansing electrodes. However, some of these electrodes may need to be subjected to additional processes in order to obtain specific desired functions.

At 12 of the electrode fabrication method 10, a base electrode is created as is known by those skilled in the art. For example, a bare polyethyleneterephthalate (PET) substrate may be prepared. Considerations in preparing the substrate include, but are not limited to, the adhesion of metal used for the base electrode to the substrate's surface, and the transfer to the final substrate upon which the electrode may be mounted which will affect the structural integrity of the electrode. Any metal or a combination of multiple metallic alloys may be used for the base electrode. For example, gold may be used in certain applications.

In alternative embodiments, a different compatible flexible substrate material and/or the thickness of the substrate material can change. For example, other types of materials that may be used rather than PET include, but are not limited to, polyamide, polyethylene naphthalate (PEN), polyurethane or any other electrically insulating polymer with a high temperature resistance. Furthermore, while a minimum thickness is desirable for the substrate layer, if it is too thin then it will be difficult for packaging.

A pattern for the metal layer may then be generated on the substrate material, which may be done using lithography, for example. The patterns that are generated have an effect on the Surface Area (SA) that is available for the sensing material as well as initial conditions for further processes such as electroplating. These initial conditions include, and are not limited to, surface potential, surface morphology, surface defect sites, defect density, crystalline structure, crystalline phase, and so on. In other words, the lithography process prepares the base electrode (for example the working electrode) in a certain way so that in the electroplating step that follows, desired nanostructures may be obtained with higher yield.]

In alternative embodiments, different patterns may be used for the metal layer such as, but not limited to, micropillars, pyramid shapes, and rectangular chambers, which are examples of periodic 3D structures.

The different patterns will affect the SA which is important since increasing the SA of the W.E. increases the area that is available for growing sensing material (also known as sensor material) and/or cause growth of one type of crystal face to dominate (e.g. 110, 111, 100) since it has been found that enzymatic activity is much stronger on specific crystalline faces such as the 110 face on gold, for example.

Referring now to FIG. 1B, shown therein is a flowchart for providing examples of implementations 20′ that may be used in the electrode fabrication method 10 of FIG. 1A. The method 20 may be done in a clean room environment. At 22, the base electrode is fabricated to have a width and length that may be in the range of about 10 μm to 100 mm and a thickness from about 1 nm to 1 mm. The base electrode may be manufactured using photo-lithography, screen printing, direct metal laser sintering or additive manufacturing (also known as additive manufacturing) such as but not limited to laser or electron beam melting additive manufacturing.

The use of a flexible substrate may be advantageous for integrating the electrode with another material that is flexible, such as a contact lens, for example. Furthermore, lithographic deposition/sputtering of a metal or multi-metal on a flexible substrate may provide a robust technology to readily translate the resulting sensors onto other wearables for applications including transdermal monitoring (e.g. wrist, EKG, body temperature, skin resistance change, etc.) and invasive methods (e.g. tattoo under the skin, electrodes in deep brain stimulating, bionic implants, etc.) Also, the flexible substrate is not limited to being flat, but may be 2D or 3D since metallic structures may be sputtered onto curved substrates (such as a contact lens).

Referring again to FIG. 1A, at 14 an intermediary layer is added to the W.E. The intermediary layer may be used not only to provide adhesion between the base electrode and the artificial functional layer, but may also be used to provide high surface area sites that may be used to grow functional elements on. If the intermediary layer is used only for adhesion, then one may use a chemical based method to create it (as in a carbon-metal hybrid such as or PPY-CNC, for example).

In at least some embodiments, the intermediary layer provides an adhesion layer in order for the artificial functional layer to have an advanced function. For example, the adhesion layer for a fractal gold sensor is a fractal gold structure and subsequent metallic layers may be formed on the adhesion layer to provide the functional layers.

The intermediary layer when placed between the base electrode 102 and the artificial functional layer may be a conductive surface for the purpose of electron transport. The interaction between the base electrode and any solution-based artificial functional layer can be either physical or chemical. A physical interaction is simpler and more cost effective; however, in some circumstances the strength of the interaction can be limited. An intermediary layer is adapted to provide greater adhesion with the artificial functional layer either through a physical or chemical interaction. The underlying mechanism of the artificial functional layer remains unaltered in the presence of the intermediary layer, and thus it can be desirable for solution-based artificial sensors.

Referring again to FIG. 1B, at 24 the intermediary layer may be created to have a width and a length of about 10 um to 100 mm and a thickness of about 1 nm to 1 mm. The intermediary layer may be manufactured using photo lithography, additive manufacturing, electroplating or chemical synthesis depending on its desired properties. For example, screen printing may be used for smaller features.

Referring again to FIG. 1A, at 16 an artificial functional layer is added to the patterned W.E. to provide the desired functionality and properties for the artificial sensor that uses the W.E. such as the type of target analytes that are to be detected, the detection sensitivity and the detection specificity. This deposition step may involve multiple metals or metal alloys for different functions such as, but not limited to, Au, Pt, and Pd. For example, artificial enzymatic functions may be obtained when favorable active sites are available for target sensing molecules to be adsorbed.

The sensing activity of the artificial sensor may be determined largely by the active sites in the artificial functional layer. By creating favorable active sites on the artificial functional layer, more efficient catalytic activities may be facilitated. Several parameters of the active sites, such as crystalline phase and dimension, for example, play a role in determining catalytic efficiency. For instance, the selection of a specific crystal phase and dimension range for nano-structures such as fractal structures may provide better specificity thus reducing sensor noise level. Meanwhile, sensitivity may be improved when most available active sites facilitate target catalytic reactions.

Referring again to FIG. 1B, at 26 the artificial functional layer may comprise an artificial enzyme that is grown on the intermediary layer. The artificial enzyme may have length, width and thickness from about 1 nm to 1 mm. The artificial enzyme may be fabricated using various techniques such as, but not limited to, electroplating, chemical synthesis, photolithography and e-beam lithography under various conditions for the deposition to fabricate the nano-micro size features that are specific as well as highly uniform. The artificial enzyme may comprise periodic nanostructures. For example, the nano-structures may be fractal structures, nano-plates, nano-pillars, nano-wires, nano-rods, nano-plates and nano-particles and the like.

In accordance with the teachings herein, by fabricating highly uniform and narrow-ranged nano-structures, a high efficiency targeting catalytic function may be achieved. For example, this may include catalyzing the oxidation reaction of various biomarkers such as, but not limited to, glucose, lactate, lipids, proteins and neutral transmitters such as dopamine.

In alternative embodiments, the material that may be used for the conductive material may change as well as the method of deposition or sputtering that is used. This is advantageous as the adhesion of the sensing material to the base electrode or the intermediary layer may change depending on the materials or deposition technique that was used. For example, different conducting materials that may be deposited on the electrode include, but are not limited to, Silver (Ag), Palladium (Pd), Platinum (Pt), Aluminum (Al), Titanium (Ti), Copper (Cu), Nickel (Ni), conducting polymers, and metal oxides. Examples of conducting polymers that may be used include, but are not limited to, PPY, PANI and PEDOT. Examples of metal oxides that may be used include, but are not limited to, Aluminum oxide, Titanium oxide and Indium Tix oxide (ITO). Different sputtering methods may be used including thermal evaporation and e-beam sputtering methods.

When the patterns for the C.E. and the R.E. are fabricated, several parameters may be varied which include the shape of the C.E. and the R.E., the material used for the C.E. and the R.E., and the SA ratio between the W.E. and the C.E. as well as the SA ratio between the W.E. and the R.E. For example, the C.E. may be about 1 to 1,000 times bigger than the W.E. and the R.E. may be 1 to 1,000 times bigger than the area of the W.E. The structural design of the W.E. relative to the C.E., the R.E., and any other electrodes that may be used in the sensor generally effects performance. For example, the surface area ratios determine the rate limiting factor during any electrochemical reaction. Thus, typically the W.E. may have a smaller surface area than the C.E. so that changes on the W.E. are reflected as a change in current between the W.E. and the C.E.

In alternative embodiments, the patterns on the C.E. and/or the W.E. may be interdigitated, be in concentric circles, or be on different layers (e.g. overlaid on top of each other).

### Creation of Various Artificial Sensors

Referring now to FIG. 2A, shown therein is a schematic showing various alternatives for fabricating artificial sensors for use in various applications. The artificial sensor is generally fabricated using methods 10 or 20 shown in FIGS. 1A and 1B respectively for creating the base electrode layer (1), the intermediary layer (2) and the artificial functional layer (3). However, the selection of certain fabrication methods, materials and sensor nanostructures result in artificial sensors that may be used in different applications.

For example, photolithography or screen printing may be used at 30 to create the base electrode (1) and chemical synthesis may be used to create the intermediary and artificial functional layers (2) and (3). At 32 the sensing material may be a hybrid carbon-based material, such as PPY-CNC for example, and the nanostructure may be nanoparticles. The result is an artificial sensor 34 that can be used to sense glucose.

As another example, screen printing may be used at 30 to create the base electrode (1), photolithography may be used to create the intermediary layer (2) and electroplating may be used to create the artificial functional layer (3). At 42, the structure used for the artificial functional layer (3) may be selected to be platinum while the intermediary layer is made of fractal AU nanostructures. Examples of materials that may be used may include a combination of Platinum and Gold or a combination of Palladium and Gold. The result is an artificial sensor 42 that can be used to sense small molecules such as but not limited to glucose and/or may be used to detect certain proteins and/or lipids.

As another example, additive manufacturing may be used at 30 to create the base electrode (1), photolithography may be used to create the intermediary layer (2) and grafting/SAM (where SAM stands for self-assembled monolayer) of 1D or 2D nanostructures may be used to create the artificial functional layer (3). At 52 the structure used for the artificial functional layer may be nitrogen doped PDA-CNC with nanoparticles. The materials that may be used include various metals (such as but not limited to Au, Pd, Ag, Pt, Ni, Cu, and Ti). The result is an artificial sensor 52 that can be used to sense small molecules 54, such as but not limited to glucose.

As another example, additive manufacturing along with conductive inks (e.g., carbon, Ag) and conductive polymers (as described previously) may be used at 50 to create the base electrode (1), photolithography may be used to create the intermediary layer (2) and e-beam lithography may be used to create the artificial functional layer (3). At 62 the structure used for the artificial functional layer may be ultrafine electrochemical membranes. The materials that may be used include metals such as, but not limited to, Au, Pd, Ag, Pt, Ni, Cu, and Ti, for example. The result is an artificial sensor 54 that can be used to sense macromolecules.

FIG. 2A shows that there may be 4 general approaches to creating artificial sensors and some of the techniques used may be interchangeable with each other since the different techniques are trying to achieve the same thing. For example, using a Fractal gold template or a CNC-based template achieves: 1) a high surface area for metal chelating, and 2) a structure for the functional layer to plate onto. As another example, the platinum 3D structure that is electroplated for the fractal Au or the platinum 0D nanoparticles that are put onto the carbon-metal CNC hybrid structure both: 1) catalyze reactions at a high rate and 2) transfer electrons to the base electrode.

Furthermore, these artificial sensors made using these different approaches may be used for long-term implants since they tend to be more stable and more robust to harsh environments.

Referring now to FIG. 2B, shown therein is a schematic drawing showing an example of a fractal structure 70 that may be used in the artificial functional layer of an artificial sensor. The fractal structure 70 may be made using gold or another metal (the material may be chosen based on the sensing application and other requirements). Initially the main branch 72 is generated. The horizontal branch 74a is the next branch to be generated. Typically, multiple horizontal branches 74a and 74b, only two of which are shown for simplicity, may be generated along the length of the main branch 72. Vertical branches may then be generated stemming from a horizontal branch such as horizontal branch 74b. The vertical branch may be oriented from an angle of 0 to 360 degrees around the horizontal branch 74b, assuming the horizontal branch 74b is the axis of rotation. A secondary horizontal branch, e.g. horizontal branch 2, may then be generated to stem from vertical branch 1 and can be oriented from an angle of 0 to 360 degrees with respect to vertical branch 1. The process of creating horizontal and vertical branches may then be repeated to create one or more fractal structures. In the example shown in FIG. 2B, three fractal structures 76a, 76b and 76c have been created.

Some parameters that may affect the growth of the fractal structure include: the concentration of various ionic species in the metal solution, the applied voltage or current, and the time of growth. New fractal structures grow as a result of an increased electric field at the tip of a previous structure. While it's difficult to control the number of repeated patterns, the general vertical length of a given fractal can be controlled. As any of the above mentioned parameters are increased in value, the fractal structure grows longer in the vertical direction and starts to develop branches. However, if too low of a value is used for any of the above mentioned parameters, the fractal structure will not form and instead there may be smaller metal islands. It should be noted that the branches do not need to be restricted to 90 degree angles as this was shown for the sake of simplicity in FIG. 2B. Accordingly, it should be understood that in alternative embodiments the angled branches may have angles other than 90 degrees. There may be embodiments in which there are multiple angled branches at different angles to form complex structures with more surface area. For example, the multiple angled branches may resemble a pine tree branch structure.

### Metal Chelating Polymer-Cellulose Nano-Crystal (MC-CNC) Artificial Biosensors

However, it may be beneficial to incorporate metal chelating polymers such as, but not limited to, PPY, PEDOT, Polydopamine and the like, due to the ability of polymers to spontaneously reduce noble metals (e.g. Ag, Pt, Au, and the like) from their salt form at room temperature without adding any capping or dispersing agents. The nature of the metal chelating polymers, as part of the intermediary layer, is to tether the functional layer (e.g. metal particles) to the intermediary layer, establishing an electrical connection between the metal particles and the base electrode. Contrary to electrical theory, a very thin layer of insulating polymer such as, but not limited to, polydopamine, can allow proper electrical coupling of the functional layer with the base electrode. The metal chelating polymers may be chosen such that they are safe for use in humans.

However, there are several disadvantages that greatly limit the use of metal chelating polymers in practical applications such as, but not limited to: poor mechanical strength resulting in poor stability due to material degradation; the agglomeration of the metal chelating polymers from typical synthesis resulting in an extremely limited surface area for sensing applications; and being indispersible or insoluble in any solvents.

However, it has been determined by the inventors that carbon-metal hybrids, including cellulose nanocrystals (CNCs) for example, may be used to overcome the aforementioned limitations of some metal chelating polymers. This is due to some of the features of carbon-metal hybrids which include: (i) strong yet lightweight, (ii) a high aspect ratio and specific surface area, (iii) enriched surface active groups, (iv) good water-dispersibility (v) low cost and abundance, and (vi) biodegradability and biocompatibility. These properties make carbon-metal hybrid materials a good candidate as a nano-template material for coating various conductive polymers thereon for various practical applications.

### Uniform Metal Nanoparticles Deposition on Carbon-Based Materials

In at least one embodiment described in accordance with the teachings herein, well-dispersed metal nanoparticles reduced on highly porous nitrogen-doped carbon nanomaterial may be used to create hybrid carbon-metal based artificial sensors.

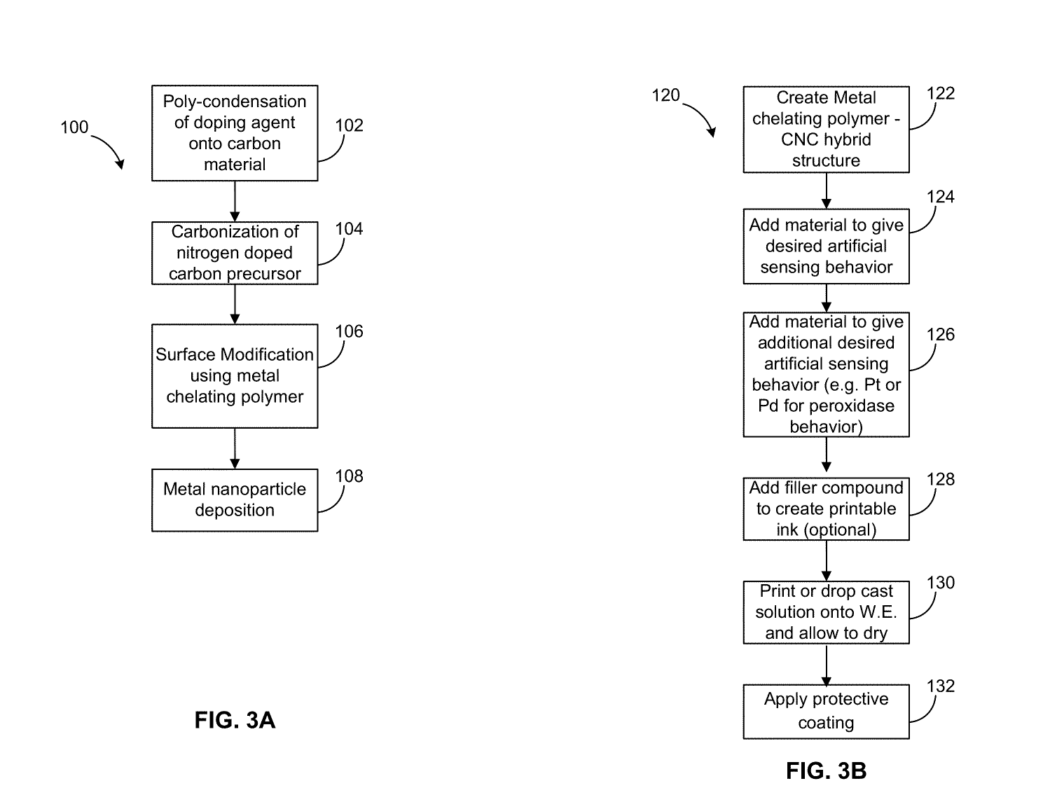

Referring now to FIG. 3A, shown therein is a flowchart of an example embodiment of an artificial enzymatic sensor fabrication method 100 to fabricate a hybrid carbon-metal based sensor. The carbon-metal hybrid material includes an intermediary layer made of metal chelating polymer (PDA, PPY, PANI, PPV etc.)-carbon based nanomaterial (CNTs, CNC, graphene, fullerene, etc.) which then tethers the artificial functional layer.

At 102, carbon materials may be used as a template to prepare nitrogen doped carbon precursors that possess high surface area and high nitrogen content. This may be done by poly-condensation, adsorption, and polymerization of a doping agent onto carbon material. As an example, melamine formaldehyde coated-CNC fibers (MFCNC) precursors may be prepared.

At 104, carbonization of the nitrogen doped carbon precursor into a nitrogen doped carbon nanomaterial may be performed. This may be done at a high temperature under an inert gas. The nitrogen doped carbon nanomaterial may then be allowed to naturally cool down.

At 106, surface modification is performed on the nitrogen doped carbon material using a metal chelating polymer to coat the nitrogen doped carbon nanomaterial with a very thin layer of metal chelating polymer. For example, N-MFCNC may be coated with a thin layer of polydopamine to form PDA-N-MFCNC.

At 108, metal nanoparticle deposition occurs which may involve adding a metal precursor along with a reducing/oxidizing agent to form metal particles with catalyst activity on the functional layer.

In an example embodiment, CNC was used as the carbon material, Melamine Formaldehyde (MF) was used as the doping agent, Polydopamine was used as the chelating polymer, and H₂PtCl₄ was used as the metal precursor. The CNC was dispersed in an aqueous medium and a precursor was prepared from a mixture of melamine and formaldehyde. The PH of the aqueous medium was adjusted to allow the formation of the precursor of MF which will in turn coat the CNC. The MF precursor solution was mixed with the CNC dispersion, or a suitable alkaline solution thereof, and the MF coating was allowed to form on the CNC material. The MF coated CNC may then be isolated by using a centrifuge or filtration.

Continuing with this example, the MF-CNC may be freeze dried, carbonized under an inert atmosphere at an elevated temperature, the resulting N-CNC may be cooled down, and then grinded into fine powders for further usage.

The N-CNC may then undergo surface functionalizing in a water-soluble substrate for further metal deposition. This may be done by dispersing well the grinded N-CNC, adjusting the PH using a Tris buffer, mixing dopamine chloride with the N-CNC solution and stirring overnight, and purifying the sample via filtration.

Metal nanoparticles may then be deposited on the N-PDCNC by dispersing well the N-PDCNC in ethylene glycol, mixing H₂PtCl₄ into the N-PDCNC solution, adjusting the PH to be alkaline, refluxing at an elevated temperature for a certain time interval to allow the reduction of the metal nanoparticles and purifying the product by filtration.

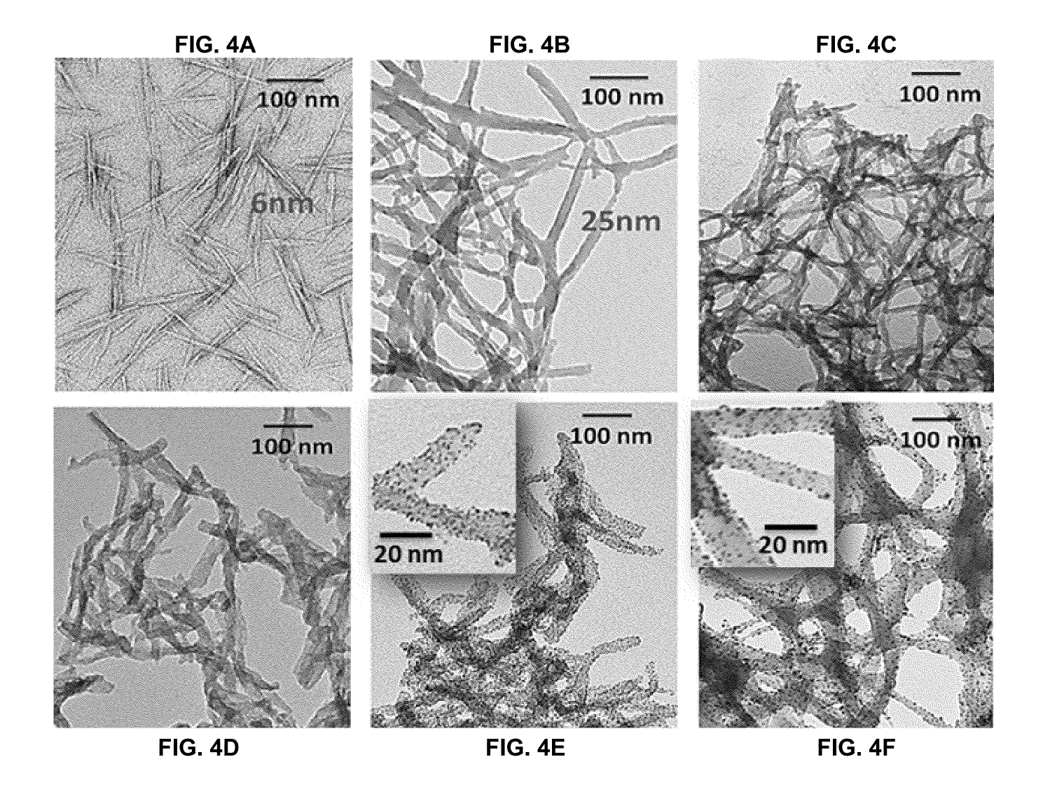

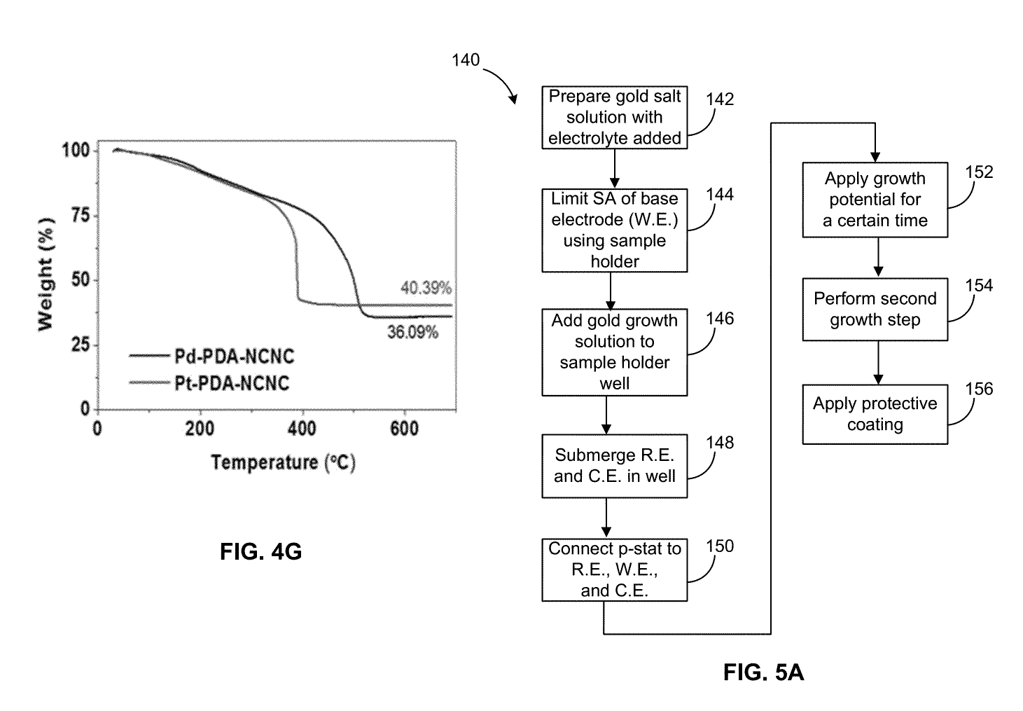

Another example of preparing MF-CNCs now follows. An example of a starting carbon material is shown in FIG. 4A. The preparing begins by mixing 10 ml 3% CNC water solution with 1.1 g of melamine and 2 mL of formaldehyde (37% in water), then adding NaOH solution to adjust the pH to 8-9, and continuing to stir at 80° C. for 1-2 h. Then, introduce an additional 40 mL deionized water was introduced into the mixture and the pH was adjusted to 4 by adding HCl solution. The reaction was kept at 60° C. for about another 2 h. The reaction was stopped by cooling it down to room temperature and purified by filtration and wash with deionized water 3 times followed by freeze drying.

The product morphology was characterized by transmission electron microscopy (TEM), and the image is shown in FIG. 4B. The CNCs after MF coating maintain the well-dispersed rod shape with slightly increased diameter due to MF coating. Thermal Gravimetric Analysis (TGA) thermal analysis for Pt—N-PDCNC and Pd—N-PDCNC with the MF-CNC in FIG. 4G shows that the most significant mass loss occurs at about less than 300° C., which is due to dehydration and elimination of the hydroxyl groups. At a higher temperature, the weight loss becomes more moderate where the evaporation of small volatile fragments and the rearrangement in the carbon frame to form graphite structure take place.

To carbonize the MF-CNC into nitrogen doped carbon fibers (N-CNC), the freeze-dried MF-CNCs were put into a quartz tube and then introduced into a high-temperature furnace, with inert gas flow operated for about 30 minutes to 1 hour to remove oxygen from the reactor at room temperature. Subsequently, the reactor was heated to a temperature of about 600˜900° C. from room temperature at a heating rate of 5 to 10° C./min. The reactor is heated to the heat treatment temperature, maintained for about 1 to 2 hours, and then naturally cooled down to room temperature. Post-heat treatment, continuous inert gas flow may be maintained until the sample cooled down. FIG. 4C shows the TEM image of the carbonized MF-CNC sample carbonized at 900° C. degrees. The rod structure remains intact up to this high temperature and even after long time grinding.

The surface modification of the N-CNC may then be done by dispersing 10 mg NCNC into 0.1% wt solution, sonicating, followed by mixing in 12 mg Tris buffer and 2 mg dopamine hydrochloride. After vigorously stirring in ambient air with an open cap for about 24 h, the reaction was stopped by repeated filtration and the N-PDCNC was obtained.

The Pt—N-PDCNC was then synthesized by dispersing about 10 mg of N-PDCNC in 20 ml ethylene glycol, which acted as the dispersant and reducing agent, and then it was well sonicated. Then 20 mg of chloroplatinic acid hexahydrate (37.5% metal basis) or 10 mg potassium tetrachloropalladate was added into the above solution and the PH was adjusted to 8.0 with NaOH. The mixture was then refluxed at about 110° C. for about 2 h followed by ultrafiltration.

Thermogravimetric analysis for Pt—N-PDCNC and Pt—N-PDCNC was conducted and the results are shown in FIGS. 4E and 4F, respectively. The high yield of 40.39% Pt loading and 36.09% Pd loading was achieved for the hybrid system.

In another example embodiment, a method of synthesizing uniformly deposited metal nanoparticles on water soluble nitrogen doped carbon fibres comprises:
1) Synthesis of Melamine formaldehyde coated cellulose nanocrystals MF-CNC;
2) Carbonizing the dried MF-CNC in a furnace under an inert atmosphere to about 500˜900° C. using a ramping temperature of about 5˜10° C./min,
3) Naturally cooling down the nitrogen doped carbon nanofiber (N-CNC) to room temperature under an inert atmosphere such as Nitrogen, Argon, etc.;
4) Grinding the N-CNC and dispersing it into a solution;
5) Coating polydopamine on the surface of N-CNC to form core-shell structured N-PDCNC which is water soluble;
6) Dispersing the N-PDCNC in ethylene glycol (which is used as the dispersant and reducing agent);
7) Mixing metal precursor with N-PDCNC solution and adjusting the PH with NaOH solution to about 8-10; and
8) Refluxing the mixture from the previous step, and reacting for a prolonged time period (about 2-3 hours) for metal reduction.

Referring now to FIG. 3B, shown therein is a flowchart of an example embodiment of an artificial enzymatic sensor fabrication method 120 for fabricating an artificial enzymatic sensor. By varying the size and shape of the nanostructures used in the artificial functional layer, different enzymatic activities may be implemented. For instance, Au nanoparticles have a glucose oxidase enzymatic function at size in the range of about 3-8 nm. However, when the size of the gold nanoparticles is about 30 nm, these particles have a peroxidase activity. This is because the catalytic function of nanostructures comes from the number of and/or size of the defect sites (e.g. active sites) on the nanoparticles/nanostructures. With carefully selected growth using a certain size and/or phase for these defect sites, specific artificial enzymatic functions may be obtained whereas other types of enzymatic functions may be discouraged. This functionality may also apply to other types of metal particles or metal oxide particles.

At 122 of the sensor fabrication method 100, a metal chelating polymer is selected for use in a CNC hybrid structure. The metal chelating polymer may be a conducting or a non-conducting polymer. Some possible choices for metal chelating polymers include, but are not limited to, PPY, Poly-thiophenes (PEDOT) and its derivatives, Polyaniline (PANI) and its derivatives, Poly(para-phenylene Vinylene) (PPV), Poly(Carbazole), Polyacetylene, Polydopamine and Polyfuran, for example. A possible rule of thumb for selecting a metal chelating polymer, may be to select a polymer that may act as a reducing agent (e.g. it may reduce something in solution thereby oxidizing itself). Generally significant reducing power is required to create a high density of nanoparticles. However, less reducing metal chelating polymers may also be able to perform this function. Accordingly, the metal chelating polymers may be chosen that display reducing power whether they possess nitrogen, sulphur or other groups.

The choice of the metal chelating polymer typically affects the biocompatibility, conductivity, stability, and overall efficiency of the metal reduction reaction. For instance, some conductive polymers have a larger threshold for oxidation and thus are able to reduce a greater number of gold particles while still maintaining metal chelating properties. The conductivity of some conductive metal chelating polymers may be recovered by exposure to acid post metal reduction. Each of these conductive metal chelating polymers may also display a unique electrochemical hysteresis effect after multiple uses. Hysteresis occurs when multiple paths exist for the chemical species or for the electron to travel through the polymer. Alternative paths can be created as a breakdown of the polymer occurs due to extended use (e.g. through many charge and discharge cycles).

At 124 of the sensor fabrication method 120, material is added to provide an artificial enzyme behaviour to the hybrid structure. For example, in the case of glucose detection, act 124 may involve adding a certain amount of Au salt to provide activity to the material that is similar to Gox. The amount of Au salt that is added will determine the particle size for the sensor. If the speed of addition is too quick or the amount of salt is too much, the formed Au will coagulate. The size of the produced Au particles will affect its ability to oxidize glucose. An AU particle in the size range of about 1-3 nm may show greater catalytic activity as compared to an AU particle in the size range of about 4-10 nm. This is due to the increase in SA to volume ratio for smaller AU particles. The amount of AU salt that may be added may depend on various conditions such as, but not limited to, electrochemical conditions for fabrication, deposition sites (base electrode) conditions (such as roughness, material, surface area), presence of other salts in the fabrication process, and concentration of the reducing agent in the fabrication process, for example. In addition, 0D nanoparticles may be advantageous as they display a higher surface area to volume ratio and thus serve as excellent catalyst for kinetically limiting reactions.

At 126 of the sensor fabrication method 120, a certain amount of peroxidase mimicking metal salt is added. For example, Pt, Pd and the like may be used. The same concept applies to these metal nanoparticles as to the Au particles in that particle size is dependent upon the amount of metal salt that is added. In addition, particles of smaller size (1-3 nm) show greater catalytic activity versus larger particles due to increase in SA to volume ratio. When gold nanoparticles are used, the amount of metal salt used is similar to the amount of gold salt that was used. It should be noted that act 126 is optional and is generally used for glucose detection.

At 128 of the sensor fabrication method 120, a filler compound may be added to the sensing material to allow ink jet printing or additive manufacturing of the material on various substrates. The filler material may be conductive, which can increase the conductivity of the otherwise insulating CNC by providing alternative routes of least resistance for electrons to flow. It should be understood that act 128 is optional. For instance, act 128 may not be done if the artificial sensor does not need to be made using additive manufacturing.

At 130 of the sensor fabrication method 120, the solution that is used to provide the desired functionality for the sensor is added to the W.E. according to a particular fabrication technique. For instance, the solution may be ink that is printed on the W.E. which may be advantageous as printing allows selective deposition of the sensing material which allows for the creation of different sensor arrangements, such as a sensor array, for example. A sensor array allows for an increase in SA. Interconnects for the sensor array can be patterned using a conductive filler. In addition, when ink is used, the ink serves as an intermediary layer, allowing for an electrical connection between the base electrode 102 and the artificial functional layer 106. The solvent in the ink can be dried if exposed to higher temperature resulting in deposition of the substituents in the ink on the base electrode 102. The interaction between the base electrode 102 and the ink deposit involves physical adhesion; however, adhesion substances can be added to the ink to allow for a chemical interaction between the ink and the base electrode 102.

Alternatively, in other embodiments, the solution may be drop cast onto the W.E. which provides less control over the pattern, but is a more cost effective solution.

If the artificial functional layer 106 displays high affinity towards the base electrode 102 then this allows for good adhesion. In other words, in the case of electrochemical growth (e.g. a fractal gold sensor), an intermediary layer may not be needed since the structure may grow molecule by molecule on the base electrode 102 and thus has already bonded tightly onto the base electrode 102. For the solution-phase sensors (e.g. PPY-CNC), the connection may be initiated by putting the solution onto the base electrode 102 and then drying the solution or allowing it to stay in the same position for a period of time. Sometimes, the base electrode 102 may be completely incompatible and no physical adhesion will occur (e.g. the solution won't stick to the surface when the water/solvent in the solution has all dried up) in which case another material can be layered on top of the base electrode 102 that will adhere to the components when the solution is dried.

The W.E. can be made using any of the following: Au, Ag, Ti, Al, Pt, Cu, Ni, alloy, conductive polymer, metal oxide (ZnO, CuO, Cu₂O), or it may be carbon based (e.g. reduced graphene oxide, Multi-wall Carbon Nanotube MWCNT)). The W.E. may be constructed on a flexible substrate (e.g. PEN, PET, polyamide, hydrogel, polymer, and the like).

At 132 of the sensor fabrication method 120, a protective coating may be applied to minimize common interferences found in bodily fluids (e.g. serum, interstitial fluid, blood, urine, sweat, tears, etc.). The artificial enzymatic biosensor may be encapsulated by one or more of a positively/negatively charged polymer, a microporous membrane, an anionic/cationic hydrogel, or a perflourinated membrane. A negatively charged polymer may limit diffusion of a buffer species (e.g. Cl—, phosphates) but may also limit diffusion of the target analyte (e.g. glucose for glucose sensing applications). A positively charged polymer may limit diffusion of protein and large molecule interferences (e.g. Ascorbic acid, Uric acid, and Acetaminophen) while promoting diffusion of the target analyte. Limiting access of interferences to the Au surface may promote increased sensitivity. For example, in glucose applications, limiting access of interferences to the Au surface may promote the kinetically limited glucose oxidation reaction and thereby allow sensing of lower glucose concentrations. Normally, small sized particles are unaffected by effects of interferences. However, an encapsulation layer may also prevent adhesion of interferences to the all of the electrodes used in the artificial sensor that are polarized.

### Multi-Metallic Fractal Artificial Sensor

In accordance with the teachings herein, at least one embodiment is provided for an artificial sensor and an associated fabrication method that utilizes platinum in the direct oxidation of glucose for continuous glucose monitoring in physiological conditions. This may be done by using a high SA fractal gold nanostructure as a template for the intermediary layer, and then depositing Pt as the functional material on the intermediary layer. In an example embodiment, the high SA fractal gold-platinum nanostructure may be prepared on a flexible gold working electrode by using a two-step electrodeposition procedure.

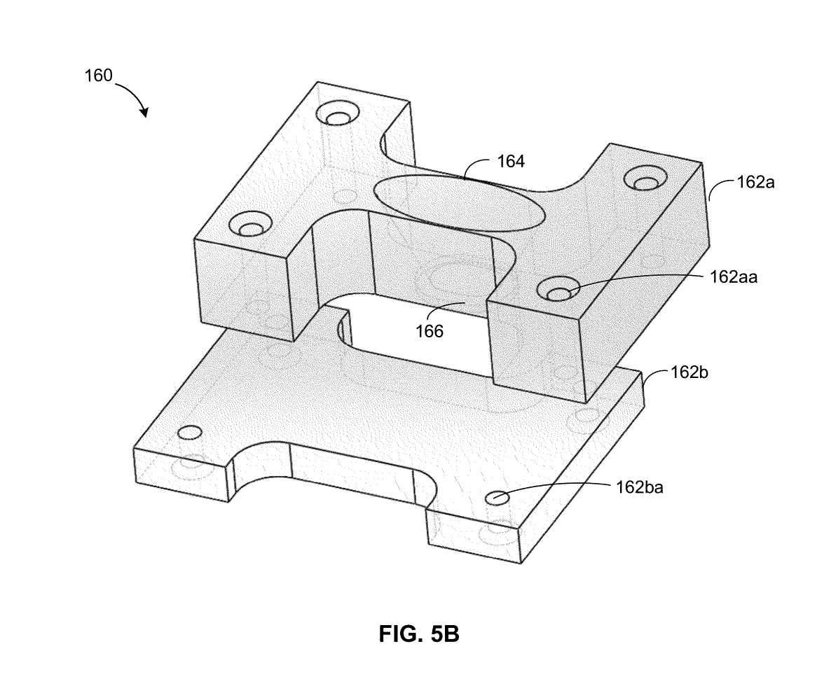

Referring now to FIG. 5A, shown therein is a flowchart of an example embodiment of an artificial enzymatic sensor fabrication method 140 for fabricating an artificial enzymatic sensor that uses a fractal gold structure as a template for the intermediary layer and a platinum structure as the artificial functional layer. While method 140 is described herein for making an artificial sensor that uses platinum in the artificial functional layer and is modified to sense glucose, the method 140 may be modified to use other metals such as but not limited to Pd, Ag, Pt, Ni, Cu, and Ti and/or to detect other target analyte such as but not limited to carbohydrates, proteins, lipids, hormones, and other essential small molecules.

At 142 of the artificial sensor fabrication method 140, a gold salt growth solution with an added electrolyte is made. The concentration of the gold salt growth solution may vary depending on the required outcome. A concentration for Au salt that is too low (e.g. concentration less than 10 mM) results in inadequate growth and thus low SA for a 1 hr growth time. A growth solution with a concentration greater than 10 mM may produce a densely packed structure that has good adhesion and high SA for a 1 hr growth period. An addition of an electrolyte such as, but not limited to, CaCl₂, Na₂SO₄, KCl, NaCl, KCH₃COOH, NH₄Cl etc., can allow for predominant growth of some crystal faces that can favor glucose binding or disfavor interference binding.

At 144 of the artificial sensor fabrication method 140, the SA of the W.E. is limited. This may be done by restricting growth of the gold fractal nanostructure to a specific location through the use of a synthesis apparatus, for example. An example embodiment of a synthesis apparatus 160 is shown in FIG. 5B that may be used for restricting growth of the gold fractal nanostructure to a specific location. Other means for synthesis may be used depending on production requirements. The synthesis apparatus 160 comprises 2 components: a top liquid holder 162a and a bottom clamp device 162b. The top liquid holder 162a has a cylindrical well 164 with a seal 166 (which in this case is an O-ring) in a desired shape at the bottom of the well 164. The O-ring may have many different shapes or sizes depending on the desired properties for the nanostructure. The O-ring seal 166 prevents leakage of growth solution and constricts its exposure to a specific area. The seal 166 may be made from rubber or another suitable material. Once the W.E. is placed on the bottom clamp device 162b and the O-ring seal 166 has been positioned on the W.E., the bottom clamp device 162b is clamped and the system 160 is sealed by the O-ring seal 166. The top liquid holder 162a and the bottom clamp device 162b may respectively comprise a plurality of channels or apertures 162aa and 162ba (only two of which are labelled for simplicity) that may receive a fastening member such as a screw or pin to releasably secure these two members 162a and 162b together. The solution is then poured down the well 164. The solution exposed surface area is defined by the cross-sectional area of the seal 166 at the bottom surface of the top liquid holder 162a and ultimately determines the charge density of the produced sensor.

In some embodiments, the synthesis apparatus 160 can be altered to allow for stirring and temperature control of the growth solution. Stirring and elevated or lowered temperatures can be used to allow growth of different morphologies for the nanostructures used in the artificial functional layer 16. The altering of the morphologies by the stirring or temperature adjustment may vary from reaction to reaction. Furthermore, the adjustment provided by stirring or temperature control may not have as large an effect as some other factors such as, but not limited to, concentration, growth voltage, template presence, for instance. As an example, the F-Au (i.e. fractal gold) sensor may be fabricated at room temperature without any stirring.

At 146 of the artificial sensor fabrication method 140, a certain amount of gold growth solution is added to the cylindrical well 164 of the top liquid holder 162a. The ratio of the volume of the gold nanostructure to the SA ratio may affect the kinetics of an analyte reaction such as a glucose reaction, for example.

At 148 of the artificial sensor fabrication method 140, the C.E. and the R.E. may be submerged in the cylindrical well of the top liquid holder 162a. The C.E. may be a Pt wire and the R.E. may be an Ag/AgCl electrode. In alternative embodiments, the C.E. and R.E. can be a variation of multiple metal layers. For example, any one of Pd, Pt, Ti, Au, Ag, Ni and Cu may be used. In addition to these metals, Iridium oxide may be used for miniature reference electrode fabrication.

The general scheme for producing a reference electrode (R.E.) is now described. Initially, a base metal layer is deposited in a desired pattern. This is followed by activation of this metal layer by addition of a redox active material. This may be achieved, for example, via drop casting, by performing an ion exchange reaction, or by electrochemically plating the active layer. The final step involves addition of a liquid reference solution as well as designing an interface between the test and the reference solution. This reference solution may be replaced with a solid-state system which eliminates the associated liquid interface, allowing for more feasible fabrication. Usually the solid state system is an ion doped membrane (e.g. Agar gel saturated with KCl or Polyvinyl chloride doped with ionic liquid). Furthermore, protective layers such as polyurethane, nafion, and silicon rubber may be added to increase stability of the solid state material.

At 150 of the artificial sensor fabrication method 140, a first lead from a potentiostat (p-stat) is coupled to the protruding W.E. surface that is not exposed to the growth solution. Second and third leads from the potentiostat are coupled to the C.E. and R.E., respectively. A potentiostat is an electronic device that may be used to control the voltage difference between several electrodes that are contained in an electrochemical cell.

At 152 of the artificial sensor fabrication method 140, a growth potential is applied for a certain amount of time. The applied voltage depends on the material used for the W.E and the desired morphology for the nano-structure. Normally, for Au and ITO working electrodes, a potential in the range of about −0.1V to −1.0V results in minor to more aggressive growth while more negative potentials of about −0.6V to −0.9V causes further side reactions to occur which produce gas bubbles, thereby altering the growth of the nanostructure. A voltage range which may be used to obtain at least satisfactory results may be about −0.1V to −1.0V for ITO and Au base electrodes with a working electrode having a gold nanostructure for the intermediary layer.

In at least some embodiments, when a fractal gold structure is used as a template, the intermediary layer may be composed of the fractal Au structure and the subsequent act 154 functionalizes this fractal Au structure with functional materials such as Pt, for example, when glucose is to be sensed by the artificial sensor.

At 154 of the artificial sensor fabrication method 140, a second growth step is performed for the creation of a bi-metallic or a multi-metallic structure through electrodeposition of a secondary growth solution. The x additional layers that may be grown may be made from at least one of Au, Pd, Ag, Pt, Cu, CuO, NiOH, and Ni on top of the current material, where x is an integer. Synergetic affects may be induced by creating a multi-metallic structure which will increase target analyte binding compared to a single structure. Some crystal faces show increased affinity for target analytes while other crystal faces have limited affinity. Bimetallic structures promote the presence of multiple crystal faces or in some cases, a single crystal face with an overall increased selectivity for the target analyte. It should be noted that platinum plating at act 154 is used to detect glucose.

At 156 of the artificial sensor fabrication method 140, a protective coating may be applied to minimize common interferences found in the environment from fouling the artificial sensor. For example, when an artificial biosensor is used in a physiological environment a protective coating may be used to prevent bodily fluids (e.g. serum, interstitial fluid, blood, urine, sweat, and tears) from fouling the artificial biosensor. For example, the artificial sensor may be encapsulated by one or more of a positively/negatively charged polymer, a microporous membrane, an anionic/cationic hydrogel, or a perflourinated membrane.

Alternatively, when glucose is the target analyte, a negatively charged polymer may be used to limit diffusion of buffer species (e.g. Cl—, phosphates) but also limiting glucose diffusion while a positively charged polymer may be used to limit diffusion of proteins and large molecule interferences (e.g. Ascorbic acid, Uric acid, Acetaminophen) while promoting glucose diffusion. Limiting access to interferences of the artificial functional layer (e.g. an AU surface) will promote the kinetically limited glucose oxidation reaction and thereby allow sensing of lower glucose concentrations. In addition, while the fractal gold nanostructures have the added ability of high SA, if the interferences are minimized then the signal to noise ratio increases.

It has been determined that the proposed gold nanostructures are able to function at a much higher sensitivity in the presence of OH species. However, alternative embodiments that use a pH modulation electrode in a 4-electrode system may provide an alkaline micro-environment that may be used to increase the glucose detection sensitivity of a gold structure. The OH species may be used to increase the sensitivity of any metal of metal oxide based electrode.

It should be noted that in the various embodiments of the W.E. described and used in accordance with the teachings herein, it has been found that the use of a flexible Au W.E. increases structural stability at more negative potentials. This is due to the increased adhesion experienced by the fractal structure as a result of the bottom Au W.E. layer. Furthermore, it has been determined, in accordance with the teachings herein, that replacement of a carbon based electrode with a flexible Au sputtered electrode may allow for stable growth at more negative potentials (−0.5->−4.0 V), which may possibly shorten total growth times.

In the various embodiments of artificial sensors described in accordance with the teachings herein, a protective layer may be added to the artificial sensors to protect against any potential interferers. The protective layer may comprise a biodegradable polymer coating such as PLGA or a co-polymer that may combine a biodegradable component with an interference exclusion component. The surface of an artificial sensor may also be coated with an anionic substance to fend off any cationic species from depositing on the artificial functional layer of the artificial sensor and fouling the artificial sensor.

In general, at least some of the artificial sensors described in accordance with the teachings herein may have at least one of the following benefits:
1. Higher sensitivity;
2. Longer life time;
3. Simpler fabrication procedure;
4. Lower working potential (which leads to increased safety and a simpler design);
5. Robust to (e.g. the artificial sensor survives) the current gold standard for cleaning contact lenses (e.g. autoclaving);
6. No oxygen requirements; and
7. More suitable for continuous monitoring.
   The artificial sensors described in accordance with the teachings herein also do not suffer from the major problem experienced by enzymatic glucose sensors which is that enzymatic glucose sensors may lose activity during standard sanitization processes involving increased temperature and other processes that are performed to the sensors when they are used for biological purposes. An example of this is the autoclaving process that is typically used in contact lens manufacturing.

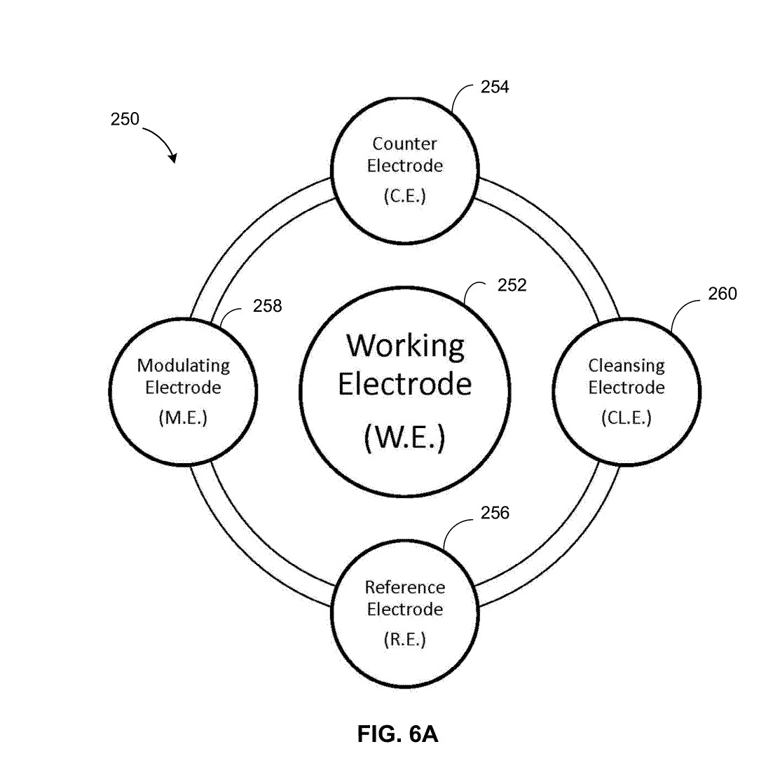

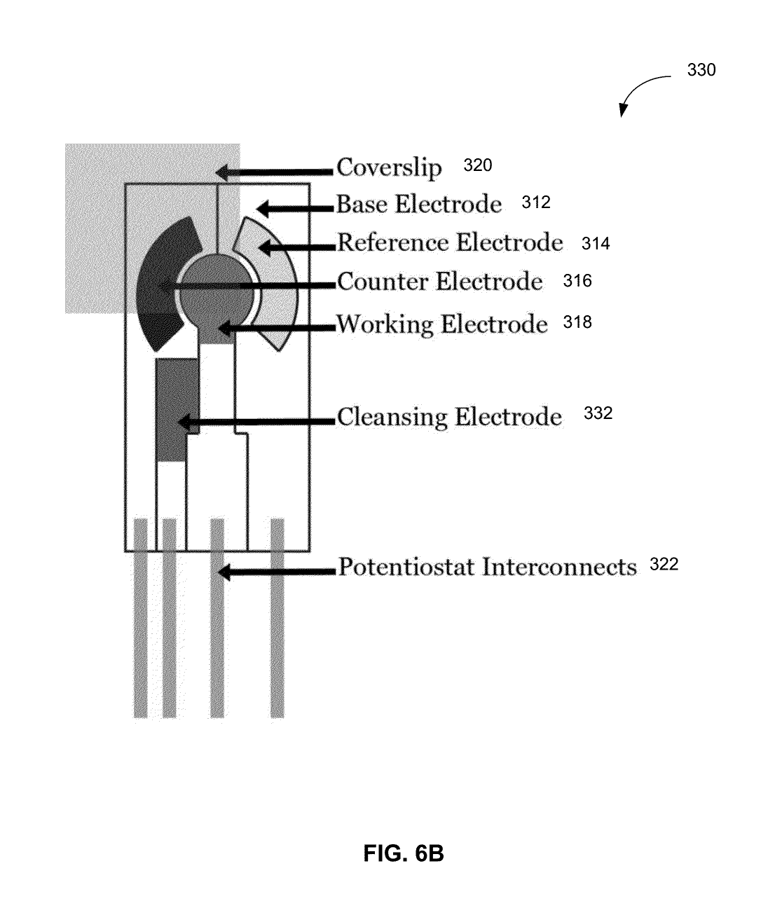

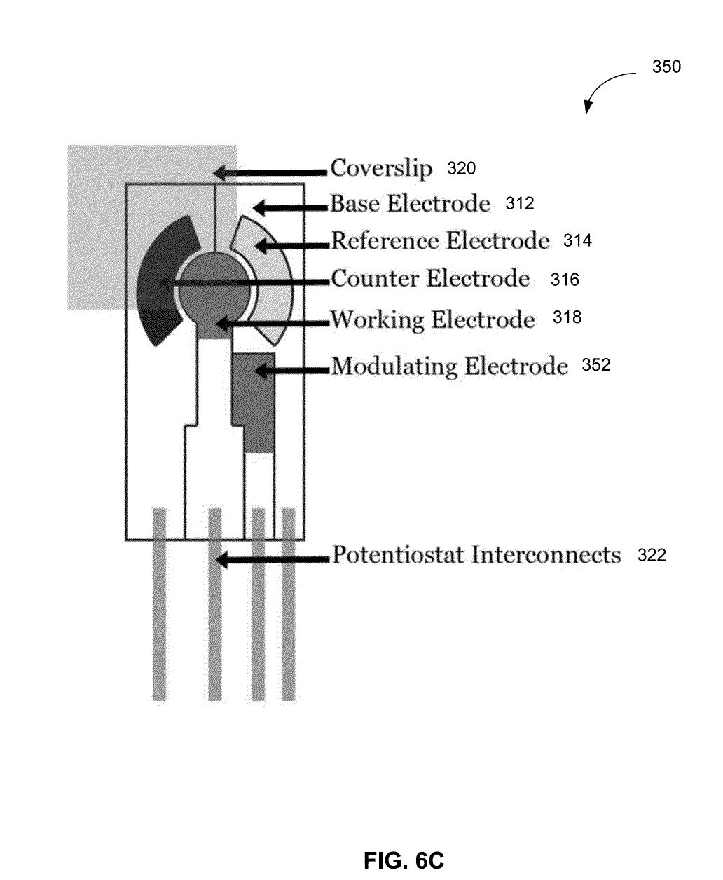

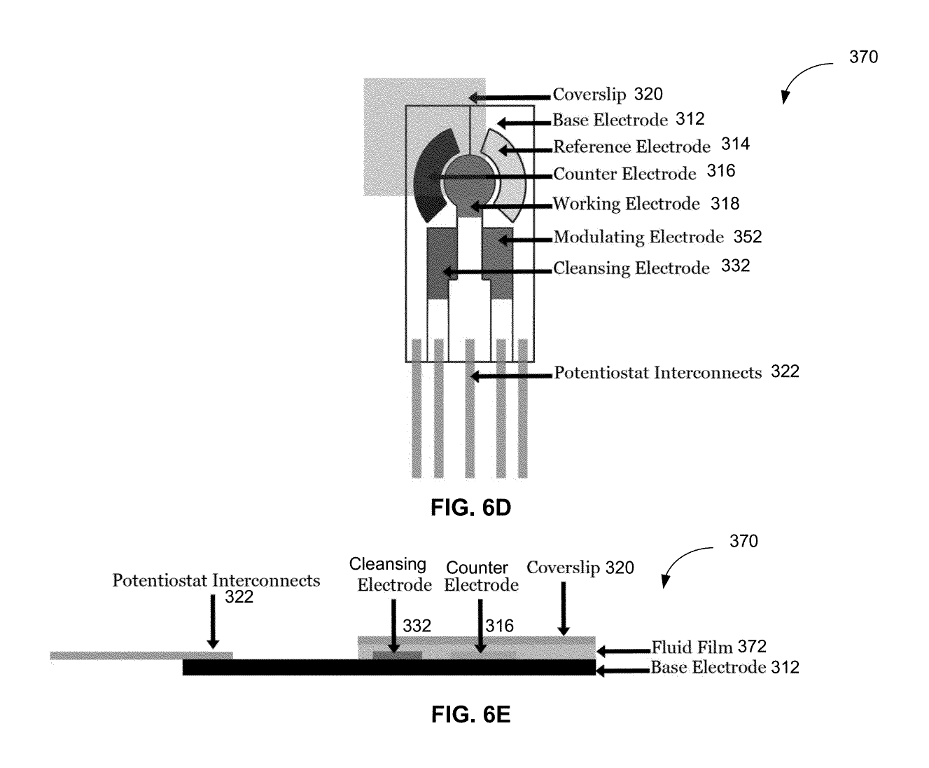

Referring now to FIG. 6A, shown therein is a schematic drawing showing an example embodiment of an electrode system 250 for an artificial sensor where the electrode system 250 may include up to five electrodes. Conventional electrode systems typically only include three electrodes: the W.E. 252, the C.E. 254 and the R.E. 256. The W.E. 252 includes the functional elements that are used to react with the target analytes for detecting the target analytes. The R.E. 256 is used to obtain measurements relative to the W.E. 252 such as current flow between the R.E. 256 and the W.E. 252. The C.E. 254 may be operated to implement a particular method of measuring the target analyte reactions occurring at the W.E. 252. For instance, the C.E. 254 may be provided with a certain excess voltage to drive any current needed so that the voltage between the R.E. 256 and the W.E. 252 is maintained at a constant level. This allows target analyte reactions at the W.E. 252 to be measured as changes in the current amplitude between the W.E. 252 and the R.E. 256.

The W.E. 252, the C.E. 254 and the R.E. provide a baseline sensitivity, specificity and stability for the artificial sensor 250. However, in accordance with the teachings herein, one or more electrodes may be added to form a 4 or 5 electrode system to improve at least one of the sensitivity, specificity and stability for the artificial sensor 250. For example, at least one of a modulating electrode (M.E.) 258 and a cleansing electrode (CL.E.) 260 may be operated with certain potentials to improve the operation of the artificial sensor 250 as will now be described.

### Micro-Environment Creation Using a Modulating Electrode in a 4-Electrode System

In accordance with the teachings herein, at least one embodiment is provided of a 4-electrode system that uses a conventional 3-electrode set-up with the addition of the M.E. 258. In this artificial sensor, the W.E. 252 provides a detection function for the detection of target analytes. The C.E. 254 facilitates current flow by the generation or donation of electrons into the artificial sensor. The R.E. 256 maintains consistent voltage and ampere conditions across the W.E. to amend for deposition or loss of materials on the W.E. The M.E. 258 may be configured to create a micro-environment with certain properties so that there may be an abundance of reagents and a desired local pH in operation. In accordance with the teachings herein, this functionality and creation of a micro-environment may be used for the detection of other target analytes.

For example, the M.E. 258 may be made of a certain material and operated at a certain voltage so that it produces certain species such as, but not limited to, O₂, H₂, H₂O₂, H₂O, and OH, that improve detection of the target analyte at the W.E. 252 since these species can selectively bind at the W.E. 252 enabling a greater specificity for the target analyte. For example, under modulating conditions: a starting molecule is catalyzed at the surface of the M.E. 258 which generates a molecule that binds specifically to the artificial functional layer of the W.E. 252 which enables easier binding of the target analyte thereby increasing the sensitivity of the artificial sensor 250.

The activity by the M.E. 258 can be considered as the M.E. 258 modulating the micro-environment around the artificial sensor 250 to affect the binding of the target analyte to the artificial functional layer of the W.E. 252. In some cases, if the micro-environment is not modulated, the target analyte will not bind to the artificial functional layer of the W.E. 252 and therefore will not be sensed.

In some cases, it may be desirable to operate the M.E. 258 so that it may change the pH level or affect other conditions of the micro-environment around the artificial sensor 250. For example, the M.E. 258 may be operated to generate hydroxide in the local environment (i.e. micro-environment). In this case, the M.E. 258 is made of a certain type of material and a certain potential is applied so that the M.E. 258 does not take up any of the generated hydroxide during operation.

Local environment control for electrochemical systems is desirable since the abundance or scarcity of a reagent or the local pH heavily dictates reaction rates. By fine-tuning the local environment condition by using the M.E. 258, desirable reactions may be better facilitated from a thermodynamic point of view. In the case of biosensing, a high (8-14) or low (1-6) pH environment may facilitate drastically different chemical activities, some of which are preferred for biosensing purposes. The production or consumption of hydroxide, oxygen, hydrogen and electrons through hydrolysis may change the local condition at the sensing sites. Modulation is achieved through sequences of electrical waveforms, or in this example, a short voltage pulse at different voltage, current, or charge conditions at the M.E. to temporarily induce micro-conditions. For example, a constant (usually negative) voltage may be applied to the M.E. 258 to produce hydroxides to alter the local pH around the artificial sensor. Desirable reactions at the W.E. are in-turn facilitated. The electrochemical system gradually equilibrates back to its original state once the sensing is done and the M.E. 258 is operated in stand-by mode (e.g. it is inactive).

Interference Species Cleansing with a Cleansing Electrode in a 4-Electrode System

An electrode system with a CL.E. 260 works towards the targeted oxidation and break down of interference species which may otherwise react with the W.E. 252 if a more favorable reaction is not present. With the CL.E. 260, impurities and interference species are broken down or denatured so that they no longer interfere with the operation of the W.E. 252.

The CL.E. 260 works with a base electrode for electrical connectivity, and may use artificial enzymes or targeting sites for break-down of interference species such as, but not limited to, uric acid, ascorbic acid, or acetaminophen, for example. Interference molecules may be either quickly consumed or decomposed through physical or chemical reactions near or on-top of the CL.E. 260. Through this process, sensitivity is enhanced because lower concentrations of target analytes may be detected without getting lost in noise that is due to the detection of an interference species. By varying the voltage, current or charge of the CL.E. 260, different interference species may be cleansed sequentially or simultaneously. Furthermore, the metal chelating polymer may be chosen so that it acts as a reducing agent as previously explained. Any residual is then either deposited on the CL.E. 260 itself, or collected in proximity of the CL.E. 260.

The cleansing electrode may be fabricated in a manner that is similar to the fabrication methods of the other electrodes. The CL.E. 260 can be made of different materials and operated at different voltage levels to provide an appropriate cleansing function for an artificial sensor that is trying to detect a particular target analyte while not detecting certain interference species. The CL.E. 260 may attract interference species to consume, degrade or eliminate them which decreases the noise level and increases the SNR of the signals measured due to the detections at the W.E. 252.

In an alternative embodiment, other cleansing mechanisms may be used. One example of another cleansing mechanism is the deposition of a thin film membrane layer with a positive or a negative charge on the artificial sensor. The membrane provides both physical filtrations of species based on pore sizes, as well as electrical repulsion of undesirable species with a similar surface charge. For example, electrochemical based sensors face interference issues due to the presence of endogenous electroactive species (e.g. ascorbic acid, uric acid, and acetaminophen) typically found in bodily fluid. Furthermore, there may be the presence of larger species such as proteins which can adhere to the electrode surface causing fouling. Generally, a negatively charged protective membrane can repel species such as ascorbic acid, uric acid, and acetaminophen due to electrostatic repulsion, thereby protecting the electrode surface. While size exclusion membranes can physically impede proteins form accessing the electrode, a negatively charged porous membrane can exhibit both activities.

In another alternative embodiment, the other cleansing mechanisms may be added to an electrode system that uses a CL.E.

In another alternative embodiment, in order to reduce interference reactions a functional group may be used which expels targeted interference species at the sensing sites. Such functional groups may be incorporated onto the W.E. 252 during its fabrication process, or incorporated onto the W.E. 252 afterwards. This may be implemented by using certain artificial enzymes on the artificial functional layer of the W.E. 252 (as described with respect to FIGS. 1B and 2.

Referring now to FIG. 6B, shown therein is a top view showing the layout of an example embodiment of an artificial sensor 310. The base electrode 312 extends along the entire rectangular area of the sensor 310. A circular W.E. 318 which provides the artificial functional layer for the artificial sensor is in the shape of a disc with an interconnect lead that are both disposed along a midline of the base electrode 312. The R.E. 314 and the C.E. 316 are shaped as partial rings that are disposed on either side of the W.E. 318 with the concave surfaces of the R.E. 314 and the C.E. 316 oriented towards the W.E. 318. An idea of the vertical layout of these components with respect to one another can be obtained by viewing FIG. 6E.

The artificial sensor 310 further comprises a cover slip 320 that is disposed at an upper left quadrant area. The cover slip 320 allows for a uniform spread of a small volume fluid across all of the electrodes (three in this case) of the artificial sensor 310. The fluid contains the analyte molecules that are to be sensed. Often, hydrophillic and hydrophobic properties of the various electrodes differ, so when the analyte is in small volumes (e.g. 1-50 μL), the fluid droplets will tend to adhere to only one of the electrodes (often the most hydrophillic). The coverslip spreads the solution across a greater SA so all of the electrodes can access the fluid.

Potentiostat interconnects 322 are used to couple a potentiostat (not shown) to the W.E. 318, R.E. 314 and the C.E. 316. The potentiostat may be used to control the artificial sensor 310 by keeping the W.E. 318 at a constant potential relative to the R.E. 314 by adjusting the current at the C.E. 316. The potentiostat typically comprises an electric circuit having operational amplifiers to provide a biasing voltage to the C.E. 316 and to make measurements between the W.E. 318 and the R.E. 314. In some cases the potentiostat also has operational amplifiers to generate control voltages to control certain electrodes such as when a modulating electrode and/or cleansing electrode are used. The artificial sensor 310 also has a cleansing electrode (CL.E.) 332 and an additional potentiostat interconnect for the CL.E. 332.

Referring now to FIG. 6C, shown therein is a top view showing the layout of another example embodiment of an artificial sensor 350 having a modulating electrode (M.E.) 352. The artificial sensor 350 is similar to the artificial sensor 310 except for the addition of the M.E. 352 and an additional potentiostat interconnect for the M.E. 352.

Referring now to FIGS. 6D and 6E, shown there are top and cross-sectional views, respectively, of the layout of another example embodiment of an artificial sensor 370 having a CL.E. 332 and a M.E. 352. The artificial sensor 370 is similar to a combination of the artificial sensors 330 and 350. The artificial sensor 370 also has additional potentiostat interconnects for the additional sensors.

It should be noted that the layouts shown in FIGS. 6B to 6E are shown as examples only and that other types of layouts are also possible depending on the application and desired properties of the artificial sensor.

In general, the M.E. 352 may be positioned such that it is in a parallel plane configuration with the W.E. 318. Furthermore, the active area of the M.E. 352 and the W.E. 318 may be in-facing such that the generated species at the M.E. 352 can be targeted to the W.E. 318. The distance between the two plates may vary from 100 μm to 1 cm. This configuration may work well for the determination of analyte concentration for single time measurements. However, it is difficult to apply this to a continuous method as the changing solution concentration may display unpredictable diffusion characteristics. In this case, the M.E. 352 may be placed on the same plane as the W.E. 318. In this case, the migration of analyte generated at the M.E. 352 is limited compared to the parallel plate configuration. However, the solution interaction with the W.E. 318 is more predictable. An increase in operation time and/or the active surface area of the M.E. 352 may facilitate an increase in the active surface area of the W.E. 318. Thus, M.E. specifications are largely dependent on the specific W.E. that they will be used with.

The CL.E. 332 may be placed on the same plane as the W.E. 318. The factor that may be varied in this case is the distance between the W.E. 318 and the CL.E. 332. In general, whether the interference species are being repulsed, attracted or converted, the CL.E. 332 may be located in closer proximity to the W.E. 318 to have a greater effect on the operation of the W.E. 318.

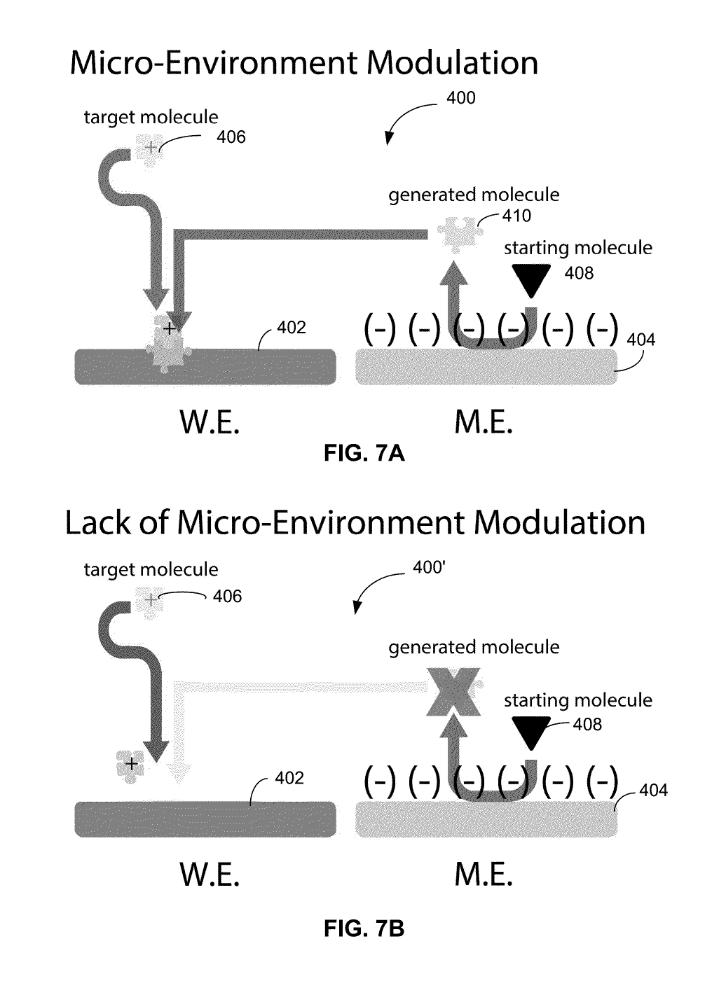

Referring now to FIGS. 7A and 7B, shown therein are schematic drawings showing various operations 400 and 400′ between a W.E. 402 and a M.E. 404 of an artificial sensor. The M.E. 404 produces certain species (e.g. O₂, H₂, H₂O₂, H₂O, and/or OH), depending on the materials used for the M.E. 404 and the operating potential, that enable and/or improve detection of a target analyte at the W.E. 402. These species generated by the M.E. 404 can selectively bind at the artificial functional layer of the W.E. 402 enabling a greater specificity for the target analyte. In FIG. 7A, under modulating conditions, a starting molecule 408 is catalyzed at the surface of the M.E. 404 to generate a molecule 410, for example, H₂O can be converted to OH—. The generated molecule 410 then binds specifically to the artificial functional layer of the W.E. 402, enabling easier binding of a target analyte 406, for example OH— can bind to metal surface for easier binding of glucose. In FIG. 7B, in which the micro-environment is not modulated, the starting molecule 408 is not catalyzed at the surface of the M.E. 404 and there are no generated molecules for binding with the artificial functional layer of the W.E. 402 and the target analyte 406 will not be sensed in this case.

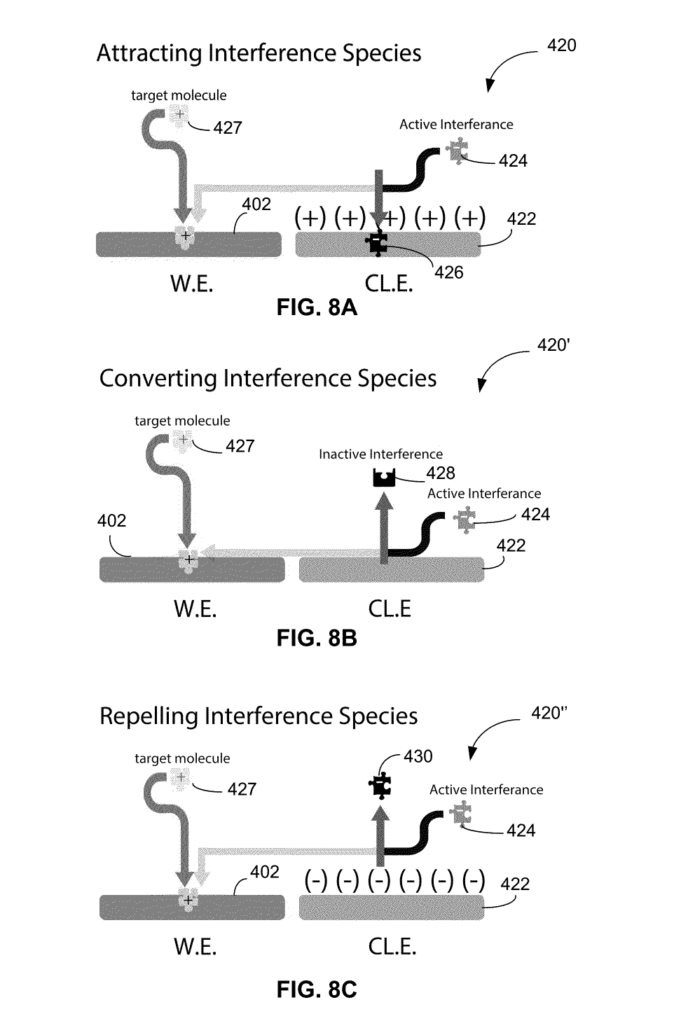

Referring now to FIGS. 8A to 8C, shown therein are schematic drawings showing various operations 420, 420′ and 420″ between a W.E. 402 and a CL.E. 422 for an artificial sensor. A certain potential, for example a −1V or a +1V potential, may be applied to the CL.E. allowing it to perform various functions including attracting an interference species (FIG. 8A), converting an interference species (FIG. 8B), and repelling an interference species (FIG. 8C), for example for species such as uric acid, ascorbic acid, acetaminophen, oxygen.

Referring now to FIG. 8A, the CL.E. 422 may be operated to attract an interference species by applying a certain charge to the electrode. For example, the CL.E. 422 may be configured to attract an interference species by applying a charge to make the CL.E. 422 positive. This will attract negative interference species and repel target analytes. The surface area of the CL.E. 422 may be selected to be very large, e.g. on the order of about 2-100 times larger than W.E., in order to draw off as many interference molecules as possible from the W.E. 402. In some embodiments, the CL.E. 422 may provide catalyzing activity for these interference species, enabling regeneration of the active sites for the CL.E. 422.

In FIG. 8A, an active interference molecule 424 attempts to bind to a site on the artificial functional layer of the W.E. 402 but the interference molecule 424 is attracted by the positively charged CL.E. 422 thereby disrupting the usual path of the interference molecule 424. In this case, the target analyte 427 is also positively charged while the interference species 424 is negatively charged so the target analyte feels an attractive force to the W.E. 402 and is repelled by the CL.E. 422.

Referring now to FIG. 8B, the CL.E. 422 may be operated to convert an interference species. For instance, a redox active interference species may be converted into a non-interfering species by selectively oxidizing or reducing the interference species. In this example, an active interference species molecule 424 attempts to gain access to the artificial functional layer of the W.E. 402 but is instead converted by the CL.E. 422 to an inactive interference species molecule 428. Meanwhile, the target analyte 427 is allowed to bind with the artificial functional layer of the W.E. 402. The target analyte 427 is positively charged while the interference species molecule 424 is negatively charged. The charges of the analyte and these molecules are taken into account as the charge attracts or repels them towards or away from the electrode.

Referring now to FIG. 8C, the CL.E. 422 may be operated to repel interference species molecules by applying a charge to the CL.E. 422 to make it negative. This will repel negative interference molecules 424 and also attract the target molecules 427 as well. However, the CL.E. 422 will not have a catalyzing function so it will not affect the concentration of the target molecules 427 in the micro-environment. This allows the target molecule 427 to successfully bind to the W.E. 402.

### Detection

Typically, there may be about 3 factors at play when choosing an appropriate detection scheme for an artificial biosensor: (1) sensing material, (2) physiological parameters and (3) any associated RF communication technology.
(1) For the sensing material, the voltage and initial equilibrium time are determined by the materials. In particular, materials that have less equilibration time are favored since intermittency between subsequent data points can be decreased. Enhancement of performance is another consideration and one possible solution may be to use detection schemes that use 4 or 5 electrode systems. Regeneration of performance is another consideration and different waveforms may be used to address this issue in accordance with the teachings herein.
(2) The physiological parameters that may be needed are the Specific Absorption Rate (SAR) based on health standards, and heat delivered to the physiological sensing location (e.g. the eye in glucose detection) which may be used to decide on intermittency of detection.
(3) RF powering and communication technology for on body wearable tech solutions may have limitations in terms of maximal level of power consumption which may cap the intermittency value (e.g. only feasible every 10 s).

Four different examples of detection schemes will now be discussed in accordance with the teachings herein. Definitions for some of the variables are in the following table.

| Parameter | Meaning |
|---|---|
| Te | time taken for equilibration to occur |
| Ts | time taken for a data point to be obtained from the sensor |
| Tst | time for which the sensor is turned off (e.g. Intermittency) |
| Tmsg | time gap between when the W.E. and the M.E. are functioning |
| | (msg = modulating sensing gap) |
| Tmt | total time when the M.E. is turned on (mt = modulation time) |
| Treg | elapsed time for regeneration voltage waveform |
| Tdata | time during which the data collection unit is functional/turned |
| | on |

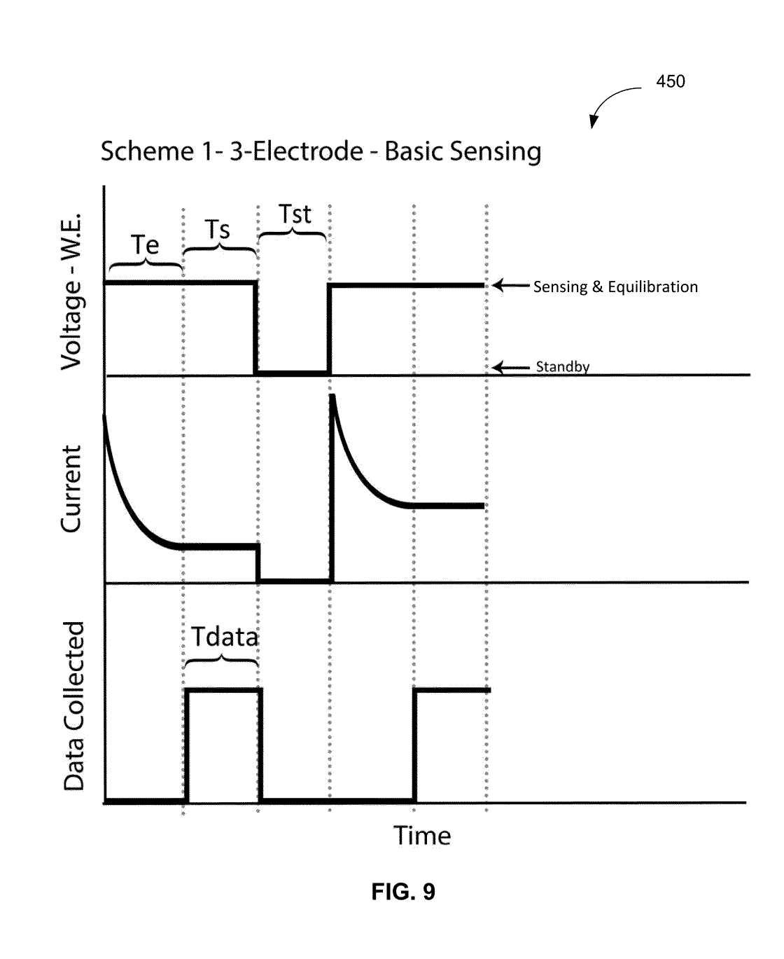

Referring now to FIG. 9, shown therein are example plots for voltage, current and collected data for an example embodiment of a detection scheme used with a 3 electrode artificial sensor. In this case, a constant voltage may be applied for sensing and equilibration. This voltage can range from −1V to 1V. The voltage used for equilibration is tied to the voltage used for sensing. Minimizing the parameters Te or Ts is beneficial since more measurement points can be determined in the same amount of time. The parameter Te may range from 1 s to 100 s while the parameter Ts may range from 1 s to 50 s. The parameter Tst determines the frequency of sensing and thus affects the SAR and heat delivered to the eye when using the sensing system on a contact lens. Ideally, data may be collected and transferred only during the sensing portion of the curve.

With the detection scheme shown in FIG. 9, first the artificial sensor is enabled and is allowed to come to equilibrium, an example of which is shown in the current signal in the middle plot. Thereafter, data is sensed from the W.E. for a period Ts. After the data is sensed, the artificial sensor is turned off for Tst. Thereafter the sensing cycle repeats itself.

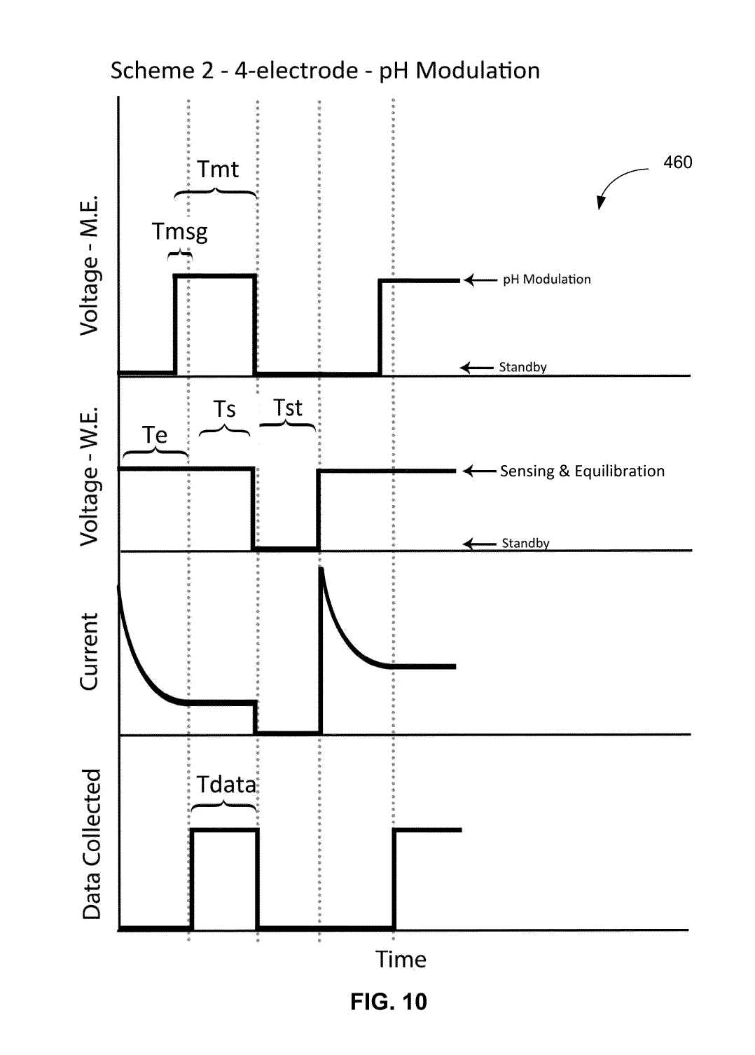

Referring now to FIG. 10, shown therein are example plots for voltage, current and collected data for an example embodiment of a detection scheme used with a 4-electrode artificial sensor including a M.E. In this scheme, a constant voltage may typically be applied for sensing and equilibration. This voltage can range from −1V to 1V. The M.E. may be used to generate OH species or to otherwise modulate the micro-environment around the artificial sensor.

When the M.E. is used to generate OH species, the OH production pulse produced at the M.E. may begin at T₀ and may last up to when the W.E. reaches standby mode (i.e. Tmt can vary from T₀ to the end of Ts). The parameter Tmsg can vary from T₀ to the beginning of Ts. Initiating Tmsg earlier than when actual sensing occurs might be used for equilibration. Since baseline current can differ between the W.E. only and the W.E.+M.E., the W.E. may be configured to only sense when the M.E. is turned on. At a minimum Tmt may overlap Ts and thus the range of values for Tmt is minimally equal to Ts. In other words, the M.E. is activated before any data is sensed by the artificial sensor and the M.E. may still be active until the end of the sensing time; i.e. the M.E. is kept active for a certain period of time such that its effects on the microenvironment are stable during the sensing period. The following parameters can cap the value of Tmt: health affects (e.g. the maximum time after which the change in microenvironment ends up affecting the macro-environment) and SARS production when the artificial sensor is used in a physiological setting.

The parameter Te may range from 1 s to 100 s while the parameter Ts may range from 1 s to 50 s. The parameter Tst determines the frequency of sensing and thus affects the SAR and the heat production. Ideally, data may be collected and transferred only during the sensing portion of the curve.

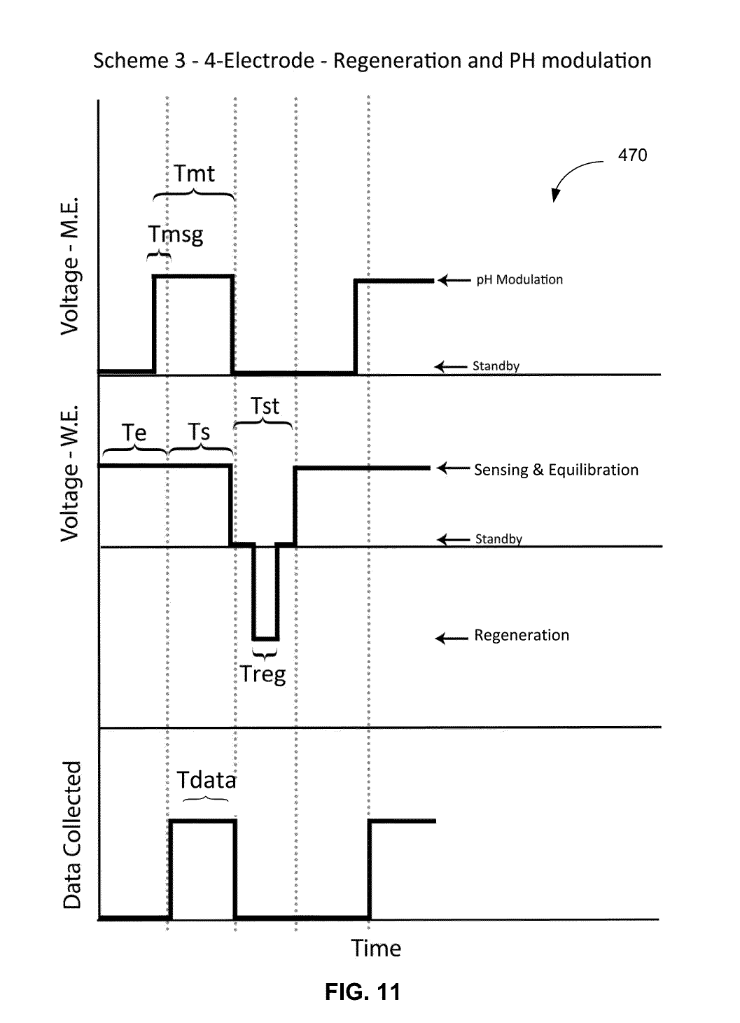

Referring now to FIG. 11, shown therein are example plots for voltages at a W.E. and a M.E. and collected data for an example embodiment of a detection scheme used with a 4-electrode artificial sensor including a M.E. for regeneration and pH modulation. All of the parameter values mentioned in relation to FIG. 10 may apply in this case. In addition, a regeneration voltage waveform (which may have a cyclic, an irregular, or a stepped shape), or in this example, a short voltage pulse may be applied to the W.E. for Treg after a short time after the end of the sensing period to remove adsorbed species from the surface of the W.E. The parameter Treg may vary from 1 ms to 100 s at negative potentials ranging from −0.1V to −4.0V. The regeneration pulse is applied during the standby period so as not to affect the data that obtained from sensing. However, the regeneration pulses may not have to be applied during every standby phase. The current generated from these regeneration pulses may not be measured. The regeneration pulses improve the longevity of the W.E. since it enables the active sites in the artificial functional layer to be used for a longer period of time since the adsorbed species are removed.

The utility of using this regeneration technique is that one pulse is used which allows for more efficient operation. This single step pulse technique is in contrast to other pulse technique which requires 2 or 3 steps.

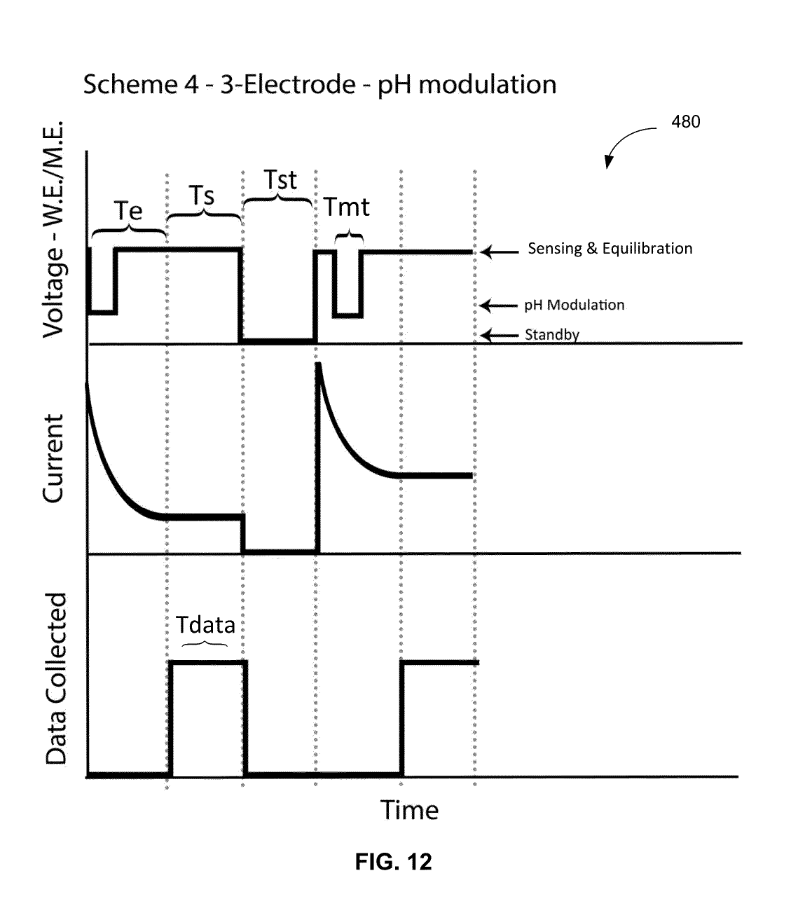

The regeneration waveform (e.g. cyclic, irregular, or stepped), or in this example, a regeneration pulse, may not have to be applied during every standby phase depending on the target analyte. For example, with glucose as the target analyte, during the sensing and equilibration phase, the glucose species are oxidized to yield the final product along with electrons that are detected. Fouling of the electrodes may occur when the glucose molecule is incompletely oxidized, thus leaving an intermediate species on the surface of the artificial sensor. This intermediate species blocks catalyst sites on the artificial sensor and is the primary reason for applying the regeneration pulse as this removes these intermediate species. This type of fouling can occur at a rapid rate or at a slow rate depending on active surface of the sensor, the applied sensing potential, the time periods Te and Ts, and the number of glucose molecules oxidized per cycle, for example. FIG. 12 shows a possible variation of the regeneration pulse where the regeneration pulse is applied during every standby phase. In this case, fouling is very frequent. In another example embodiment, the regeneration pulse can be applied before the 1^{st} to the 1000^{th} sensing cycle during the standby phase.

Referring now to FIG. 12, shown therein are example plots for voltage, current and collected data for an example embodiment of a detection scheme used with a 3 electrode artificial sensor where the W.E. is used for sensing and modulation. All of the parameter values discussed in relation to FIG. 9 apply here. In addition, a pH modulation waveform may be applied to the W.E. so that it operates as a M.E. to generate OH species. The modulation waveform may occur before the W.E. begins sensing (i.e. before the Te phase) such that the OH species are able to add a synergetic effect towards selectivity of the artificial sensor for the target analyte. The parameter Tmp may range from 0 s to 100 s.

When there is a 4 or more electrode system that uses a CL.E. then, since the purpose of the CL.E. is to reduce noise during the sensing of data, the CL.E. is turned on when the artificial sensor is not in standby mode. When a voltage is applied to the CL.E., the CL.E. provides oxidation or decomposition to eliminate interference species during sensing. The CL.E. is functional during the equilibration and sensing period (i.e. Te and Ts). The voltage of the CL.E. may be in a series of pulses or it can be a long pulse.

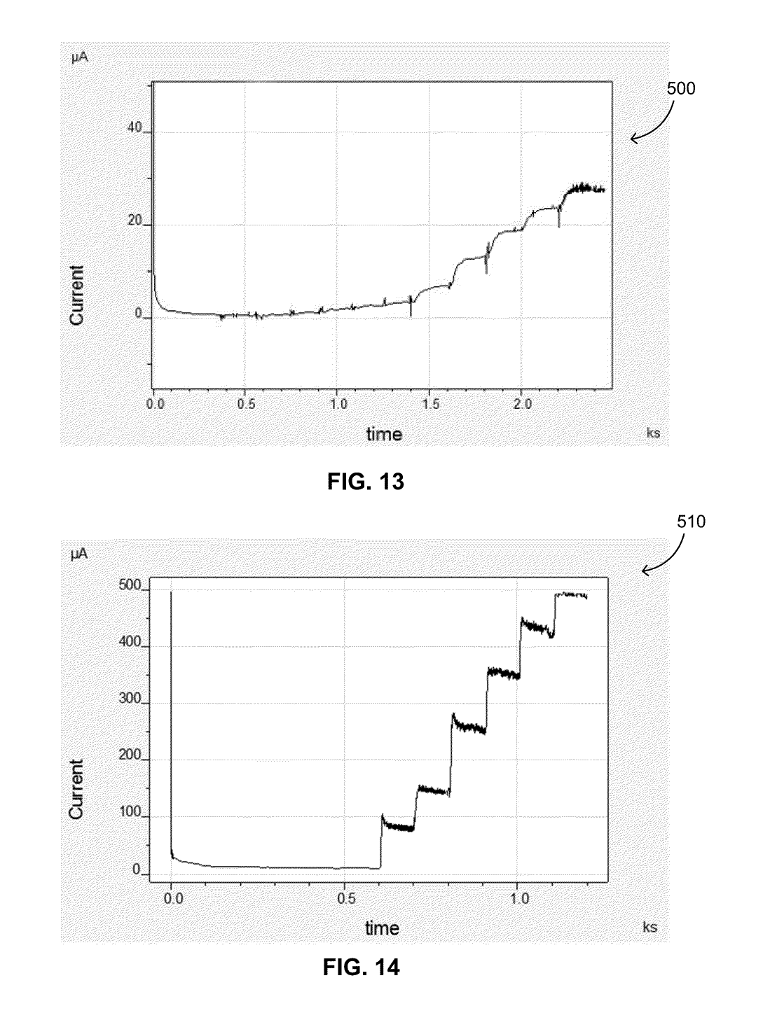

Referring now to an example embodiment of an artificial sensor which uses gold in the artificial functional layer to detect glucose, it has been found that gold's ability to bind and directly oxidize glucose changes based on the orientation of the growth (i.e. which crystal face is the predominant crystal face) and the total active SA. Normally, in neutral physiological conditions, the specificity of gold for glucose versus other species in a buffer solution is fairly low. For example, referring to FIG. 13, shown therein is an example of an Amperometric detection curve for the detection of glucose using a fractal gold intermediary layer with a gold functional structure in neutral pH conditions (i.e. alkaline media (+0.1M NaOH in Phosphate Buffer Saline (PBS) solution)). The gold structure may be plated from a HAuCl₄ salt as per act 154 in FIG. 5A. The PBS-based solution is used to try to emulate certain physiological conditions. The following concentration additions were made: 0.5 mM @ 300 s, 1 mM @ 400 s, 2 mM @500 s, 3 mM @600 s, 4 mM @ 700 s, 5 mM @ 800 s, and 6 mM @ 900 s.

However, as can be seen in FIG. 13, in neutral pH conditions, the lower glucose concentrations in the range of 0.1-1 mM are not resolved (e.g. the change between 0.1 mM and 0.2 mM cannot be quantitated) since the gold nanoparticles in the artificial functional layer do not prefer binding glucose versus other buffer species in a solution. However, when the glucose concentration increased in the solution at 500 s, the gold nanostructures in the artificial functional layer were then able to bind to the glucose and detect it. This is because the relative amount of glucose was now greater versus the buffer species. Because the first step direct glucose oxidation (direct indicates that the electrons are not passed to an intermediary compound but instead are captured by the oxidizing material i.e. Au) is the adsorption of glucose to the gold nanoparticles in the artificial functional layer, the glucose needs to compete with other species in solution (e.g. Cl—, PO₄₃—, uric acid, ascorbic acid, etc.) to bind to the gold surface. This first step is used to continue the complex 2e− oxidation of glucose. This reaction is under kinetic control since the complex steps need to reach the end. However, compared to a diffusion controlled reaction, such as ascorbic acid oxidation (a common bodily fluid interference), the signal from a kinetic controlled reaction increases many folds when the catalytic SA is increased.

Referring now to FIG. 14, shown therein is an example of an amperometric detection curve using a gold intermediary layer with a gold functional structure in alkaline pH conditions (Phosphate Buffer Saline (PBS) +0.1 M NaOH). This may be achieved by using a M.E. to generate OH. In alkaline media, the lower glucose concentration range is well resolved (i.e. the change between 0.1 mM and 0.2 mM can be quantitated) and the steps can be easily seen. This is because the hydroxides (OH—) in solution bind to the gold particles and synergistically assist the gold particles to specifically bind with glucose molecules. In FIG. 14, amperometric detection using a gold intermediary layer with a gold functional structure in alkaline (+0.1M NaOH in PBS) was done with the following concentration additions: 0.5 mM @ 600 s, 1 mM @ 700 s, 2 mM @ 800 s, 3 mM @ 900 s, 4 mM @ 1000 s and 5 mM @ 1100 s. An example calibration curve for a fractal gold structure with an Au functional layer in alkaline pH conditions (with NaOH) is shown in FIG. 14. FIG. 14 shows the fast response time and highlights the synergetic effect of OH which can be applied to other types of artificial sensors that may be detecting other types of target analytes.

In an alternative example embodiment, of an artificial sensor which uses a bimetallic structure comprising fractal-gold template and platinum that are both in the artificial functional layer to detect glucose, it has been found that modification of pH below or above physiological levels is not required to achieve high sensitivity.

The use of artificial sensors, in accordance with the teachings herein, for detecting glucose in vivo is important since management of diabetes is complex as the level of blood glucose entering the bloodstream is dynamic. Variation of insulin in the bloodstream that controls the transport of glucose out of the bloodstream also complicates diabetes management. Blood glucose levels are sensitive to diet and exercise, but may also be affected by sleep, stress, smoking, travel, illness, menses, and other psychological and lifestyle factors unique to individual patients.

The dynamic nature of blood glucose and insulin, and all other factors affecting blood glucose, often require a person with diabetes to forecast blood glucose levels. This allows therapy in the form of insulin or oral medications or both to be timed to maintain blood glucose levels in an appropriate range. However, management of diabetes is often highly intrusive because of the need to consistently obtain reliable diagnostic information, follow prescribed therapy, and manage lifestyle on a daily basis. Daily diagnostic information, such as blood glucose concentration, is typically obtained from a capillary blood sample with a lancing device and is then measured with a handheld blood glucose meter. Interstitial glucose levels may be obtained from a continuous glucose sensor worn on the body. However, some of the artificial sensors described herein may be incorporated with a platform that interfaces with a person's physiology with minimal intrusion to obtain the glucose measurements in real time throughout the day.

In general, it should be noted that the sensor technology described herein may be used, or modified in some cases, to sense a large variety of target analytes that are biomarkers found within the precorneal tear such as, but not limited to, acids, carbohydrates, proteins, enzymes, lipids, immunoglobulins, mediators, hormones, medication, and recreational drugs for example.

Acids of interest may include, but is not limited to, ascorbic acid, biocarbonate, and uric acid for example. For example, a fractal gold artificial sensor with a platinum functional layer in accordance with the teachings herein may detect ascorbic and uric acid as these species are redox active. These 2 acids are normally considered interference when sensing glucose.

With regards to carbohydrates, the artificial sensor embodiments described herein may be used to detect common carbohydrates without alteration to the structure. Carbohydrates of interest may include, but are not limited to, fructose, glucose, sucrose, galactose, maltose, and lactose, for example.

With regards to proteins, the artificial sensors described herein that incorporate a nanoparticle necklace may be used to detect proteins. Proteins of interests may include, but are not limited to, lysozyme, lipocalin, lactoferrin, tear-specific pre-albumin (TSP), lipocalin, epidermal growth factor (EGF), albumin, antiproteinases, secretory component (SC), glycoproteins, orosomucoid, transferrin, ceruloplasmin, and immunoglobulins, for example.

Enzymes may be detected in a similar fashion as proteins. Some enzymes of interest may include, but are not limited to, hexokinase, phosphoglucoseisomerase, phosphofructokinase, aldolase, triose phosphate isomerase, enolase, pyruvate kinase, glyceraldehyde-3-phosphate dehydrogenase, phosphoglyceratemutase, lactic dehydrogenase (five isoenzymes), pyruvate dehydrogenase, citrate synthase, aconitase, isocitric dehydrogenase, α-ketoglutarate dehydrogenase, succinyl-Coa-synthase, succinate dehydrogenase, fumarase, matalate dehydrogenase, glucose 6-phosphate dehydrogenase, 6-phosphogluconolacotne, 6-phosphogluconate dehydrogenase, ribulose-5-phosphate isomerase, ribulose-5-phosphate epimerase, transketolase, transaldolase, transketolase, lysozyme, amylase, proteases, antiproteases, peroxidase, plasminogen activator, lysosomal acid hydrolases.

With regards to lipids and hormones, artificial sensors described herein with nanostructures may be used since the nanostructures can react with any redox active species and since most lipids and hormone have redox active components within their structure, these lipids and proteins can be detected. In some embodiments, these artificial sensors may be modified to add other metal layers to the artificial functional layer to enable sensing of various components. In some embodiments, these artificial sensors may be further modified to include various repellant layers or attractive layers to improve target analyte specificity. Some lipids of interest may include, but are not limited to, wax esters, sterol esters (mainly cholesterol), polar lipids, hydrocarbons, diesters, triglycerides, free sterols, free fatty acids, etc. ‘Hormones of interest may include, but are not limited to. catecholamines, endorphins, and dopamine, for example.

Immunoglobulins of interest may include, but are not limited to, sIgA (IgA), IgG, and IgM, for example. Since immunoglobulins are a type of protein species, the detection technique(s) described for proteins may also be applicable to immunoglobulins.

Mediators of interest include prostaglandins, for example. Prostagladins fall into the category of lipids and so the detection technique(s) described for lipids should be applicable for prostaglandins.

Medications of interest may include ibuprofen, acetaminophen, alrex, betaxon, besivance, cosopt, lucentis, metformin, sulfonylureas, meglitinides, thiazolidinediones, Adriamycin, adruicil, Cytoxan, ethyol, and leukeran, for example.

Recreational drugs of interest may include psychedelics, opium, LSD, barbiturates, benzodiazepines, amphetamines, ecstasy (MDMA), cocaine, heroin, cannabis, for example.

The various embodiments of artificial sensors described in accordance with the teachings herein may provide several benefits including at least one of:
1. Prolonged lifetime due to regeneration;
2. Reduces cost of medical devices as a result of infrequent replacements due to the longer lifetime; and
3. Inert to harsh temperature, humidity, and oxidizing species such as acids and peroxides.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without departing from the embodiments described herein, the general scope of which is defined in the appended claims.

1. An artificial sensor for sensing target analytes, wherein the artificial sensor comprises:
a working electrode that is configured to provide a detection signal indicating detection of the target analytes during a sensing phase;
a reference electrode that is configured to provide a reference level for measurements made at the working electrode;
a counter electrode that is configured to provide a current source or a current sink for the working electrode during use; and
at least one additional electrode that is configured to improve signal to noise ratio of the detection signal when provided with a control voltage during use.

2. The artificial sensor of claim 1, wherein the at least one additional electrode comprises a modulating electrode that is configured to modify local conditions around the working electrode when provided with the control voltage during use.

3. The artificial sensor of claim 2, wherein the modulating electrode is configured to increase a number of rate-limiting reagents in the micro-environment when provided with the control voltage during use.

4. The artificial sensor of claim 3, wherein the rate-limiting reagents comprise at least one of O₂, H₂, H₂O₂, H₂O, and OH.

5. The artificial sensor of claim 2, wherein the modulating electrode is configured to generate a desired local pH in the micro-environment by consuming or producing hydroxide when provided with the control voltage during use.

6. The artificial sensor of claim 2, wherein the control voltage comprises sequences of electrical waveforms at different voltage, current, or charge conditions to temporarily modify the local conditions of the micro-environment.

7. The artificial sensor of claim 1, wherein the at least one additional electrode comprises a cleansing electrode that is configured to breakdown or consume an interference species when the control voltage is applied during the sensing phase to reduce an effect of the interference species on the working electrode.

8. The artificial sensor of claim 7, wherein the cleansing electrode comprises artificial enzymes or targeting sites to breakdown the interference species.

9. The artificial sensor of claim 7, wherein the voltage, current or charge of the cleansing electrode is varied during use to cleanse different interference species sequentially or simultaneously.

10. The artificial sensor of claim 7, wherein the cleansing electrode is configured to receive a charge to attract interference species and to repel target analytes.

11. The artificial sensor of claim 7, wherein the cleansing electrode is configured to convert an interference species to a non-interference species by selectively oxidizing or reducing the interference species.

12. The artificial sensor of claim 1, wherein the at least one additional electrode comprises a modulating electrode and a cleansing electrode wherein the modulating electrode is configured to modify local conditions around the working electrode when provided with the control voltage during use and the cleansing electrode is configured to breakdown or consume an interference species when the control voltage is applied during the sensing phase to reduce an effect of the interference species on the working electrode.

13. A method of sensing target analytes using an artificial sensor, wherein the method comprises:
providing a detection signal at a working electrode indicating detection of the target analytes thereabout during a sensing phase;
providing a reference level at a reference electrode for providing a baseline for measurements made at the working electrode;
providing a current source or a current sink at a counter electrode to provide or remove current from the working electrode during use; and
applying a control voltage to at least one additional electrode to improve signal to noise ratio of the detection signal.

14. The method of claim 13, wherein the method comprises using a modulating electrode as the least one additional electrode to modify local conditions around the working electrode when provided with the control voltage.

15. The method of claim 14, wherein the method comprises using the modulating electrode to increase a number of rate-limiting reagents in the micro-environment when provided with the control voltage during use.

16. The method of claim 14, wherein the method comprises applying the control voltage to the modulating electrode to generate a desired local pH in the micro-environment by consuming or producing hydroxide.

17. The method of claim 14, wherein the method comprises applying sequences of electrical waveforms at different voltage, current, or charge conditions in the control voltage to temporarily modify the local conditions of the micro-environment.

18. The method of claim 12, wherein the method comprises using a cleansing electrode as the at least one additional electrode to breakdown or consume an interference species when the control voltage is applied during the sensing phase to reduce an effect of the interference species on the working electrode.

19. The method of claim 18, wherein the method comprises providing a charge at the cleansing electrode to attract interference species and to repel target analytes from the cleansing electrode.

20. The method of claim 18, wherein the method comprises using the cleansing electrode to convert an interference species to a non-interference species by selectively oxidizing or reducing the interference species.

21. The method of claim 13, wherein the method comprising using a modulating electrode and a cleansing electrode as the at least one additional electrode wherein the modulating electrode is used to modify local conditions around the working electrode when provided with the control voltage during use and the cleansing electrode is used to breakdown or consume an interference species when the control voltage is applied during the sensing phase to reduce an effect of the interference species on the working electrode.

22. The method of claim 13, wherein the method comprises applying a regeneration voltage waveform to the working electrode after the end of the sensing phase to remove adsorbed species from a surface of the working electrode.

23. The method of claim 13, wherein the regeneration voltage waveform is not applied after the end of every sensing phase.

24. A working electrode for an artificial sensor that detects target analytes, wherein the working electrode comprises:
a base electrode that is conductive;
an intermediary layer disposed adjacent to the base electrode;
comprising growth sites; and
an artificial functional layer that is coupled to the intermediary layer and configured to provide an artificial sensing function, the artificial functional layer comprising at least one artificial catalyst.

25. The working electrode of claim 24, wherein the intermediary layer comprises a fractal metal nanostructure that provides a template with increased surface area for the at least one artificial catalyst in the functional layer.

26. The working electrode of claim 24, wherein the fractal structure comprises one or more metals.

27. The working electrode of claim 24, wherein the artificial functional layer comprises a multi-metallic nanostructure where the nanostructure enhances working electrode performance.

28. The working electrode of claim 24, wherein the artificial functional layer is configured for detecting a desired type of target analyte at a desired detection sensitivity and detection specificity by including multiple metals or metal alloys into the artificial functional layer.

29. The working electrode of claim 28, wherein the functional layer comprises platinum nanostructures that detect glucose.

30. The working electrode of claim 24, wherein the intermediary layer comprises a metal chelating polymer and a carbon nanomaterial, wherein the metal chelating polymer covers the carbon nanomaterial and the carbon nanomaterial is doped or un-doped.

31. The working electrode of claim 30, wherein the metal chelating polymer comprises a conductive polymer.

32. The working electrode of claim 30, wherein the metal chelating polymer comprises an insulating polymer that is thin enough to allow electron transfer thereacross.

33. The working electrode of claim 32, wherein the insulating polymer comprises polydopamine.

34. The working electrode of claim 30, wherein the intermediary layer comprises nanoparticles that are produced on the metal chelating polymer, are dense and are similar in size range to provide a more homogeneous distribution of catalyst sites in the functional layer and improve detection.

35. The working electrode of claim 34, wherein the nanoparticles comprise gold nanoparticles having a size in the range of about 3-8 nm to detect glucose.

36. The working electrode of claim 30, wherein the metal chelating polymer comprises at least one of polypyrrole (PPY), Polydopamine, Poly-thiophenes (PEDOT) and its derivatives, Polyaniline (PANI) and its derivatives, Poly(para-phenylene Vinylene) (PPV), Poly(Carbazole), Polyacetylene, and Polyfuran.

37. The working electrode of claim 30, wherein the intermediary layer comprises PPY-CNC or PDA-CNC disposed adjacent to the base electrode.

38. A method for creating an artificial sensor for detecting target analytes, wherein the method comprises:
creating a base electrode;
creating an intermediary layer on the base electrode; and
creating an artificial functional layer on the intermediary layer.

39. The method of claim 38, wherein the method comprises growing a fractal metal nanostructure in the intermediary layer to provide a template with increased surface area for at least one artificial catalyst in the functional layer.

40. The method of claim 39, wherein the method comprises growing a multi-metallic structure in the artificial functional layer to improve working electrode performance by performing a second growth step through electrodeposition of a secondary growth solution.

41. The method of claim 40, wherein the additional layers are grown from at least one of Au, Ag, Ti, Al, Pt, Cu, Ni, alloy, a conductive polymer, and metal oxide.

42. The method of claim 41, wherein the method comprises growing platinum nanostructures in the artificial functional layer to detect glucose.

43. The method of claim 38, wherein the act of creating the intermediary layer comprises:
selecting a doped or undoped carbon nanomaterial; and
coating the carbon nanomaterial with a chelating metal polymer.

44. The method of claim 43, wherein coating the carbon nanomaterial comprises providing an insulating polymer that is thin enough to allow electron transfer thereacross.

45. The method of claim 43, wherein the act of creating the artificial functional layer comprises forming metal particles having catalyst functionality in the artificial functional layer.

46. The method of claim 40, wherein the act of forming metal particles comprises adding a metal precursor with a reducing agent or an oxidizing agent.

47. The method of claim 38, wherein the method further comprises adding a protective coating to the artificial sensor to reduce a number of common interferences at the artificial sensor.

48. The method of claim 38, wherein creating the intermediate layer comprises selecting a conducting polymer for integration in a Cellulose Nano-Crystal (CNC) hybrid structure.

49. The method of claim 38, wherein creating the artificial functional layer comprises adding material to a solution to provide enzyme mimicking behaviour to the hybrid structure.

50. The method of claim 38, wherein the additional material comprises one of Au, Ag, Ti, Al, Pt, Cu, Ni, metal alloys, conductive polymer, metal oxide and carbon based material.

51. The method of claim 50, wherein the carbon based material comprises one of reduced graphene oxide, graphene, fullerene, and a Multi-wall Carbon Nanotube (MWCNT).

52. The method of claim 39, wherein the fractal metal nanostructure comprises a gold nanostructure, a surface area of active sites on the functional layer is limited, and a ratio of a volume of the gold nanostructure to the limited surface area is selected to affect the kinetics of an analyte reaction with the target analytes.
